(19) United States
(12) Patent Application Publication
(10) Pub. No.: US 2024/0287128 A1
(43) Pub. Date: Aug. 29, 2024

(54) EXTREMELY FAST SOLID PHASE SYNTHESIS

(71) Applicant: YISSUM RESEARCH DEVELOPMENT COMPANY OF THE HEBREW UNIVERSITY OF JERUSALEM LTD., Jerusalem (IL)

(72) Inventors: Chaim GILON, Jerusalem (IL); Mattan HUREVICH, Jerusalem (IL); Moshe BENTOLILA, Moshav Tkuma (IL); Israel ALSHANSKI, Har Gilo (IL); Johnny NAOUM, Haifa (IL)

(21) Appl. No.: 18/253,146

(22) PCT Filed: Nov. 17, 2021
(86) PCT No.: PCT/IL2021/051368

§ 371 Date: May 16, 2023

Related U.S. Application Data
(60) Provisional application No. 63/114,556, filed on Nov. 17, 2020.

(51) Int. Cl. C07K 1/04

(52) U.S. Cl. CPC C07K 1/045

(57) ABSTRACT

The present disclosure generally relates to the field of solid phase synthesis and methods for synthesizing peptides and employing solid phase synthesis.

### TECHNICAL FIELD

The present disclosure generally relates to the field of solid phase synthesis and methods for synthesizing peptides employing solid phase synthesis.

### BACKGROUND

There are many applications of peptide drugs. There are about 100 approved peptide drugs on the market among them several blockbusters such as the somatostatin analog octreotide. The most popular and dominant segments of peptide drugs include HIV treatment, diabetes, cancer treatment, with applications in the fields of neurological disorders, such as Alzheimer's and Parkinson's disease, as well as autoimmune diseases, such as Lupus, Rheumatoid Arthritis and Uveitis also constantly arising.

Three main methods are used to prepare large quantities of peptides in the pharmaceutical industry: recombinant technologies; solution phase chemical synthesis; and solid phase peptide synthesis (SPPS). SPPS is the manufacturing procedure of choice for peptides due to the low cost of raw materials and economy of scale. Solid-phase chemistry approaches are faster and less expensive for manufacturing from gram to 100 Kg scale of relatively short peptides (typically up to about 50 amino-acid long) and are better suited to early-stage clinical development.

The SPPS process is a multi-step chain process involving two main types (i.e., stages or steps) of reactions: (i) removal of temporary protecting group (deprotection) and (ii) coupling of an amino acid. These reactions take place sequentially one after the other, with intermediate washings taking place for removal of unreacted reagents and soluble side-and by-products. In general, in solid phase syntheses a sequence of building blocks is constructed over an insoluble polymeric resin having linking units reactive towards the building blocks. Since some building blocks, such as amino acids comprise at least two functional groups reactive towards one another (i.e., the amino and carboxylate groups), one group, typically the amino group is protected beforehand, such that the free carboxylate group reacts with the linking unit, rather than with another present amino acid. For continuation of the amino acid chain, deprotection of the amino groups is preformed, thereby producing a resin having the first amino acid, free for reaction with the consecutive N-protected amino acid. The process repeats until a desired number of building blocks is achieved. The purpose of using an insoluble resin is to enable easy workup procedure of simply washing all excess soluble reagents, as well as side-and by-products, while remaining with the desired product attached to the insoluble resin. The last synthetic step is, therefore, removing the side-chain protecting groups and cleaving the building block chain (the peptide) and isolating it from the insoluble resin.

Thus, solid phase syntheses of a peptide, typically include the following synthetic steps: (a) swelling of functionalized resin polymeric beads; (b) coupling a first protected monomeric unit (typically, protected amino acid) to the functionalized resin; (c) washing the excess of the first protected monomeric unit; (d) deprotecting the resin linked to the first monomer formed in step (b) using a deprotection reagent, thereby forming a resin linked to the first deprotected monomer; and (c) washing the excess of the deprotection reagent. Steps (b)-(c) are then cyclically repeated, with the subsequent protected monomers, thereby forming resin-peptide. Thus, steps (b)-(c) are regarded as cycles, where the total solid phase synthesis typically includes a plurality of cycles. Lastly, the desired product peptide is cleaved from the resin and is separate therefrom, thereby obtaining the final polymeric product.

The resin used for solid-phase synthesis is typically an insoluble polymer, modified with chemically reactive functional groups. These functional groups are chemically suitable to be coupled with an amino acid, which is to be incorporated into the target product peptide. Said first monomer, as well as the other sequential monomers, typically includes at least two functional groups, which are reactive towards one another. However, the placement of protecting group on one of these functional groups restricts the transformation to coupling of the protected monomeric unit into the resin, thereby forming a resin, which is initially linked to the first monomer; and after a number of similar cycles, linked to a construct of monomers. Typically, each protected monomer in the sequence of cycles is used in excess (with respected to the resin-bound molecules) for ensuring the completion of the reaction. Typically, the deprotection reagent is also used in excess (with respected to the resin-bound monomer molecules) for ensuring the completion of the reaction.

Since the invention of SPPS by Bruce Merrifield in 1963 (Merrifield, R. B. (1963) J. Am. Chem. Soc. 85, 2149-2154) many methods were introduced to improve that technique. Most of these methods are based on chemical modification of coupling reagents (Pattabiraman et al. Nature (2011) 480, 471-479). Other improvements include thermal methods e.g., microwave and heating (Collins et al. (2014) Org. Lett. 16, 940-943). Nevertheless, very few investigations were made on the influence of the hydrodynamic parameters on the yield and side reactions in SPPS.

Secondary and tertiary structures of polypeptides are particularly sensitive to shearing forces. When exposed to shear, the secondary and/or tertiary structure of a peptidic molecule can be irreversibly altered, potentially resulting in loss of biological activity. The primary structure, i.e., the amino acid sequence, may be also destroyed or interrupted if the peptidic molecule is subjected to sufficient shear force. Considering that, method of synthesizing peptides generally utilizes procedures that minimize the exposure of peptidic molecules to shear stress. For example, EP0503683 discloses a “vortex” agitation mode that prevents resin agglomeration and allows total fluid-resin interaction without the use of impeller type mechanical agitation. According to this publication, with mechanical agitation, the shear and resin abrasion caused by the impeller can fracture the resin beads into smaller and smaller particles which can eventually clog the filters, thus forcing interruption of the synthesis process. With the vortex agitator there are no impeller type shear or abrasive effects on the resin beads.

In his seminal paper (Merrifield, R. B. (1963) J. Am. Chem. Soc. 85, 2149-2154) Merrifield used a magnetic stirrer for mixing the beads of polymeric resin. It turned out that this method breaks the resin bead to small particles that blocks the sinter glass filter and prevent filtration. To avoid this problem Merrifield invented a manually operated apparatus consisting of a reaction vessel, in which the reactants are mixed by shaking rather than by stirring (Merrifield, R. B. Solid Phase Synthesis (Nobel Lecture), Angewandte Chemie International Edition (1985) 24, 799-892). Since then, production of peptides is generally restricted to employing gentle mixing methods, such as vortex, nitrogen stream, rotation in rotary evaporator rotors, agitation by rocking etc. In large scale Solid phase synthesis (SPS) glass reactors are typically employed with a mechanical stirrer at low rpm for gentle agitation of the resin beads. The impeller of such stirrer is specially designed for agitation with very low shear rate.

WO 2019/175867 discloses a method for performing at least one cycle of solid phase synthesis of an organic molecule, the method comprising the steps of: (a) providing a reactor comprising a reaction chamber and a stirring apparatus comprising an impeller having at least two blades rotatable about an axis; (b) inserting beads of functionalized polymeric resin and at least one solvent into the reactor to provide a reaction mixture, wherein the reaction mixture is in contact with the rotatable blades; (c) inserting at least one protected monomeric organic molecule and at least one coupling agent into the reaction chamber and spinning the impeller, thereby forming a coupling product of the protected monomeric organic molecule and the resin; (d) washing excess of said protected monomeric organic molecule; and (e) inserting at least one deprotecting reagent into the reaction chamber and spinning the impeller, thereby removing at least one protecting group from the coupling product, forming a coupling product of the deprotected monomeric organic molecule and the resin, thereby completing a cycle in the solid phase synthesis of a polymeric organic molecule; wherein the spinning of the impeller in at least one of steps (c) and (e) is performed for a period of time, at a rotational rate of at least 600 rounds per minute, while maintaining a shear rate of at least 3·10³ sec⁻¹, optionally wherein steps (c) to (c) are repeated a plurality of cycles. WO 2019/175867 discloses the synthesis of the tri-peptide Fmoc-L-His-Phe-Gly-NH₂ by repeating steps (c)-(c) twice at ambient temperature. The period of time required for performing the coupling reaction of step (c) was 30-90 minutes and period of time required for performing the deprotection reaction of step (c) was 10-20 minutes.

There is a need for improved methods for synthesizing peptides in large scale with higher efficiency and speed and lower side-reactions, to provide greener and accessible alternative to the state of the art.

### SUMMARY OF THE INVENTION

The present invention provides improved methods of solid phase synthesis of oligomers, such as peptides or hybrids thereof. The invention is based in part on the unexpected finding that solid phase synthesis using the combination of elevated reaction temperature and high shear forces and steering speed, do not harm the solid support or synthesized molecules but improves synthesis efficiency such as synthesis time, yield and purity. Moreover, it was found the combination of the elevated temperature and the high shear properties result in an extremely fast solid phase synthesis. Specifically, it was found that such reaction conditions for forming peptides are leading to extremely fast solid phase peptide synthesis (EF-SPPS).

The following embodiments and aspects thereof are described and illustrated in conjunction with systems, tools and methods, which are meant to be exemplary and illustrative, not limiting in scope.

According to one aspect, the present invention provides a method for solid phase stepwise synthesis of peptides, including modified peptides (e.g., glycopeptides and phosphopeptides), and peptide hybrids, comprising at least one mixing step which involves (a) heating and (b) employment of high shear force within the synthesis mixture, wherein a solid-phase resin is in contact with a stirring apparatus at all synthesis steps of the stepwise synthesis.

According to some embodiments, the method comprises the steps: i. providing a reactor comprising a reaction chamber and a stirring apparatus comprising an impeller having at least two blades rotatable about an axis; ii, inserting beads of functionalized polymeric resin and at least one solvent into the reactor to provide a reaction mixture, wherein the reaction mixture is in contact with the rotatable blades; iii, inserting at least one reactant into the reaction chamber; and iv. spinning the impeller for a period of time, at a rotational rate of at least 600 rounds per minute, while maintaining a shear rate of at least 3·10³ sec⁻¹, wherein the temperature within the reaction chamber is in the range of 40° C., to 100° C., thereby performing at least one step of the solid phase synthesis.

According to some embodiments, the process further comprises heating the reaction chamber to allow the temperature range in step iv. According to some embodiments, step iv. is performed at a temperature in the range of 50° C., to 90° C. According to some embodiments, step iv. is performed at a temperature in the range of 45° C., to 95° C. According to some embodiments. step iv. is performed at a temperature of at least 35° C., at least 40° C., at least 45° C., at least 50° C., at least 55° C., at least 60° C., at least 65° C., at least 70° C., at least 75° C. or at least 80° C. Each possibility represents a separate embodiment. According to some embodiments, step iv. is performed at a temperature of no more than 100° C., no more than 95° C., no more than 90° C., no more than 85° C., no more than 80° C. or no more than 75° C. Each possibility represents a separate embodiment.

It was found by the inventors of the present invention that the combination of (a) high shear rate of mixing the reaction mixture and (b) reaction temperature above the ambient temperature, results in improved yield and reduced amount of side products. Specifically, it is very uncommon in the art to apply high shear stress on reaction solid phase synthesis reaction mixtures. This since the solid phase beads, which comprise the reactants of reaction mixtures are believed to be sensitive the high shear stress. It was further believed that combining elevated reaction temperatures with the high shear stress would be yet even more detrimental to the structural stability of the beads. However, it was surprisingly found that the combination of elevated reaction temperatures with the high shear mixing properties results in ultra-fast reactions, whereby side products and bead degradation are essentially suppressed.

According to some embodiments, step iv. is performed for a period of time in the range of 5 seconds to 2 minutes. According to some embodiments, step iv. is performed for a period of time in the range of 5 seconds to 105 seconds. According to some embodiments, step iv. is performed for a period of time in the range of 5 seconds to 90 seconds. According to some embodiments, step iv. is performed for a period of time in the range of 5 seconds to 75 seconds. According to some embodiments, step iv. is performed for a period of time in the range of 5 seconds to 75 seconds. According to some embodiments, step iv. is performed for a period of time in the range of 5 seconds to 60 seconds. According to some embodiments, step iv. is performed for a period of time in the range of 5 seconds to 45 seconds. According to some embodiments, step iv. is performed for a period of time in the range of 5 seconds to 30 seconds. According to some embodiments, the period of time is no more than 180 seconds, no more than 170 seconds, no more than 160 seconds, no more than 150 seconds, no more than 140 seconds, no more than 130 seconds, no more than 120 seconds, no more than 110 seconds, no more than 100 seconds, no more than 90 seconds, no more than 80 seconds, no more than 70 seconds, no more than 60 seconds, no more than 50 seconds, no more than 45 seconds, no more than 40 seconds, no more than 35 seconds or no more than 30 seconds. Each possibility represents a separate embodiment. According to some embodiments, the period of time is at least 5 seconds, at least 10 seconds, at least 15 seconds, at least 20 seconds or at least 25 seconds. Each possibility represents a separate embodiment. According to some embodiments, the period of time is about 30 seconds.

According to some embodiments, the reactor further comprises a heating assembly and an enclosure, which defines an internal cavity and comprises the reaction chamber. According to some embodiments, the enclosure has an internal face facing the internal cavity and an external face. According to some embodiments, the at least two blades are disposed within the internal cavity. According to some embodiments the heating assembly is in contact with the external enclosure face.

According to some embodiments, the heating assembly is surrounding the enclosure. According to some embodiments, the enclosure is substantially cylindrical and is surrounded by the heating assembly.

According to some embodiments, the heating assembly is selected from a circulating fluid bath and a heating jacket. According to some embodiments, the heating assembly is a heating jacket. According to some embodiments, the heating assembly is a circulating fluid bath. According to some embodiments, the heating assembly is configured to transfer heat to the external enclosure face from a heated fluid circulating in the heating assembly. According to some embodiments, the heating assembly is configured to transfer heat to the external enclosure face from a heating coil within the heating assembly. According to some embodiments, the heating assembly is not a microwave heater.

According to some embodiments, the internal cavity defined by the enclosure is divided by a top internal cavity portion and a bottom internal cavity portion by a semipermeable disc having a plurality of pores therein. According to some embodiments, the internal cavity is divided by a semipermeable disc to a top internal cavity portion and a bottom internal cavity portion, wherein the semipermeable disc is having a plurality of pores therein. According to some embodiments, the top internal cavity portion has a greater volume than the bottom internal cavity portion. According to some embodiments, the semipermeable disc is in contact with the internal enclosure face. According to some embodiments, the semipermeable disc is flat and round, and the enclosure has a matching round cross section, wherein the semipermeable disc is in contact with the internal enclosure face throughout said cross-section.

According to some embodiments, the semipermeable disc is permeable to fluids. According to some embodiments, the semipermeable disc is impermeable to the beads of the functionalized polymeric resin. According to some embodiments, the semipermeable disc is permeable to fluids and impermeable to the beads of the functionalized polymeric resin. According to some embodiments, the semipermeable disc is positioned within the enclosure such that fluid communication is enabled between the top internal cavity portion and the bottom internal cavity portion, and passage of the polymeric resin beads is prevented between the top internal cavity portion and the bottom internal cavity portion. According to some embodiments, the process comprises placing the polymeric resin beads on the semipermeable disc, such that they are within the top internal cavity portion.

According to some embodiments, the rotatable blades are disposed within the top internal cavity portion. According to some embodiments, the polymeric resin beads are within the top internal cavity portion during step iv. According to some embodiments, upon inserting the beads of the functionalized polymeric resin in step ii., the beads are confined within the top internal cavity portion. According to some embodiments, at least a portion of the solvent is within the top internal cavity portion during step iv. According to some embodiments, a part of the solvent is within the top internal cavity portion, and another part of the solvent is within the bottom internal cavity portion during step iv. According to some embodiments, the reaction chamber is within the top internal cavity portion. According to some embodiments, in step iv. the shear rate and the temperature range are maintained within the top internal cavity portion. According to some embodiments, the reaction chamber is within the top internal cavity portion, and wherein in step iv, the shear properties and the temperature range are maintained there within. The term “shear properties” as used herein, refers to the shear rate, the shear stress and the shear force.

According to some embodiments, the semipermeable disc is a glass fritted disc.

The terms “fritted disc” and “fritted filter disc” are interchangeable and refer to flat discs typically used in chemical laboratories for separation between insoluble solids and liquids from a mixture. These discs differ in their porosity, with nominal maximum pore size ranging from 4 microns (for fine fritted discs) to 220 microns (for extra course fritted discs).

It is to be understood by the skilled in the art that polymeric resin beads used in solid phase peptide synthesis are typically large enough to be blocked by course fritted discs. Thus, liquid compositions (e.g., aggregates and dispersions), containing such polymeric resin beads can be filtered through course fritted discs resulting in separation between the liquid medium and the solid beads. However, it is to be understood by the skilled in the art that more fine discs may also be employed for filtering polymeric resin beads.

According to some embodiments, the fritted disc has nominal maximum pore size (NMPS) of at least 40 microns. The term “nominal maximum pore size” is known in the art and refers to a specification of porous filter discs, which defines the diameter of the largest pores within the disc. According to some embodiments, the fritted disc has an NMPS in the range of 40 microns to 100 microns.

According to some embodiments, the reactor further comprises a conduit extending from a proximal conduit end to a distal conduit end, wherein the proximal conduit end is connected to a portion of the enclosure, which encloses the bottom internal cavity portion. According to some embodiments, each one of the proximal conduit ends and the distal conduit end is an open end. According to some embodiments, the distal conduit end is located out of the enclosure. According to some embodiments, the reactor further comprises a valve configured to monitor flow of fluids within the conduit. According to some embodiments, when the valve is in an open state, fluid communication between the bottom internal cavity portion and the distal conduit end is enabled. According to some embodiments, when the valve is in a closed state, fluid communication between the bottom internal cavity portion and the distal conduit end is prevented.

According to some embodiments, the distal conduit end is directly or indirectly connected to an inert gas cylinder, or a vacuum pump. According to some embodiments, the distal conduit end is directly or indirectly connected to at least one of an inert gas cylinder and a vacuum pump. According to some embodiments, the distal conduit end is directly or indirectly connected to an inert gas cylinder. According to some embodiments, the distal conduit end is directly or indirectly connected to a vacuum pump. According to some embodiments, the valve is configured to monitor flow of inert gasses and/or liquids. According to some embodiments, the conduit is configured to enable flow of inert gasses and/or liquids. According to some embodiments, the method comprises flow of inert gas or gasses and/or liquid or liquids into the reactor through the conduit. According to some embodiments, the method comprises flow of inert into the reactor through the conduit.

According to some embodiments, step iii. of inserting at least one reactant into the reaction chamber entails providing the reactant through the conduit. According to some embodiments, the reactant is provided as a flowable composition, and step iii. entails flowing the reactant through the conduit. According to some embodiments, the reactant is provided as a liquid composition, and step iii. entails flowing the liquid composition through the conduit into the reaction chamber. According to some embodiments, the reactant is provided as a liquid solution, and step iii. entails flowing the liquid solution through the conduit into the reaction chamber.

According to some embodiments, the distal conduit end is directly or indirectly connected to an inert gas cylinder, wherein the valve is configured to monitor flow of inert gasses, or wherein the distal conduit end is connected directly or indirectly to a vacuum pump, wherein the valve is configured to monitor flow of liquids.

According to some embodiments, the reactor further comprises a three-way bidirectional conduit extending from a proximal conduit end to a first distal conduit end and to a second distal conduit end. Three-way conduits are known in the art, examples thereof can be seen in FIGS. 1A and 1B. Bidirectional conduits are conduits, which enable flow of fluids therethrough in both directions, i.e., from the proximal conduit end to the distal conduit end and vice versa. The direction of flow is typically based on fluid pressure differential, which may be applied by a pump or by pressurized gas.

According to some embodiments, the conduit comprises at least three ends. According to some embodiments, each of the conduit ends is an open end, such that fluid communication may be enabled between the proximal conduit end and each of the distal conduit ends.

According to some embodiments, the proximal conduit end is connected to a portion of the enclosure, which encloses the bottom internal cavity portion. According to some embodiments, the first distal conduit end is connected directly or indirectly to an inert gas source. According to some embodiments, the second distal conduit end is connected directly or indirectly to a vacuum pump. According to some embodiments, the second distal conduit end is connected indirectly to a vacuum pump, wherein the method further comprises drawing a liquid composition comprising a reaction product, by product or excess reagent from the reactor, through the conduit, by application of vacuum. According to some embodiments, the second distal conduit end is connected indirectly to a vacuum pump; step iii. comprises inserting at least one reactant in excess amount into the reaction chamber; the method further comprises, after step iv., evacuating a liquid composition comprising an excess of the at least one reactant from the reactor, through the conduit, by application of vacuum through the conduit. According to some embodiments, upon the step of evacuation the reacted beads are maintained within the reactor. According to some embodiments, upon the step of evacuation the reacted beads are maintained within the reaction chamber, wherein the reaction chamber is essentially devoid of additional reagents. According to some embodiments, the reactor further comprises a three-way valve configured to monitor flow of both liquids and gasses through the conduit.

According to some embodiments, the reactor further comprises a three way bidirectional conduit extending from a proximal conduit end to a first distal conduit end and to a second distal conduit end, wherein the proximal conduit end is connected to a portion of the enclosure, which encloses the bottom internal cavity portion, wherein the first distal conduit end is connected directly or indirectly to an inert gas source, wherein the second distal conduit end is connected directly or indirectly to a vacuum pump, wherein the reactor further comprises a three way valve configured to monitor flow of both liquids and gasses.

According to some embodiments, the second distal conduit end is indirectly connected to a vacuum pump through a vacuum trap. Vacuum traps are known chemical lab glassware. An example is shown in FIG. 1B.

According to some embodiments, the present invention provides the reactor as detailed above. According to some embodiments, there is provided a reactor comprising: a stirring apparatus, a heating assembly and an enclosure. According to some embodiments, the enclosure encompassed the reaction chamber. According to some embodiments, the stirring apparatus comprises an impeller having at least two blades rotatable about an axis, and configured to spin the blades at a rotational rate of at least 600 rounds per minute (rpm). According to some embodiments, the blades are disposed within the enclosure. According to some embodiments, the enclosure defines an internal cavity and has an internal face facing the internal cavity and an external face, wherein the heating assembly is in contact with the external enclosure face. According to some embodiments, the heating assembly is surrounding the enclosure and is selected from a circulating fluid bath and a heating jacket. According to some embodiments, the internal cavity is divided by a semipermeable disc to a top internal cavity portion and a bottom internal cavity portion, wherein the semipermeable disc is having a plurality of pores therein. According to some embodiments, the rotatable blades are disposed within the top internal cavity portion. According to some embodiments, the reactor further comprises a conduit extending from a proximal conduit end to a distal conduit end, wherein the proximal conduit end is connected to a portion of the enclosure, which encloses the bottom internal cavity portion, and the distal conduit end is located out of the enclosure. According to some embodiments, the reactor further comprises a valve configured to monitor flow of fluids within the conduit. According to some embodiments, reactor further comprises a three way bidirectional conduit extending from a proximal conduit end to a first distal conduit end and to a second distal conduit end, wherein the proximal conduit end is connected to a portion of the enclosure, which encloses the bottom internal cavity portion, wherein the first distal conduit end is connected directly or indirectly to an inert gas source, wherein the second distal conduit end is connected directly or indirectly to a vacuum pump, wherein the reactor further comprises a three way valve configured to monitor flow of both liquids and gasses. Additional features of the reactor of the present invention are elaborated herein above when referring to the solid phase synthetic method and in the detailed description section of the present disclosure.

According to some embodiments, the method of the present invention comprises the steps of: i. providing the reactor; ii, inserting beads of functionalized polymeric resin and at least one solvent into the enclosure to provide the reaction mixture in contact with the rotatable blades; iii, inserting at least one reactant into the reaction chamber within the enclosure; and iv. spinning the impeller for a period of time, at a rotational rate of at least 600 rounds per minute (rpm), to maintain the shear rate of at least 3·10³ sec⁻¹ within the reaction chamber, wherein the temperature within the reaction chamber is in the range of 40° C., to 100° C. According to some embodiments, the method further comprises activating the heating assembly to bring the reaction mixture within the enclosure to a temperature in the range of 40° C., to 100° C., in step iv.

It is to be understood that that in addition to step iv., other steps of the present method may be conducted at the elevated temperatures of step iv. According to some embodiments, during step ii., the temperature within the reaction chamber is in the range of 40° C., to 100° C. According to some embodiments, during step iii., the temperature within the reaction chamber is in the range of 40° C., to 100° C. According to some embodiments, during each one of steps ii.-iv. the temperature within the reaction chamber is in the range of 40° C., to 100° C.

According to some embodiments, the method comprises the steps of: i. providing the reactor; ii, inserting the beads of functionalized polymeric resin into the top internal cavity portion and inserting the at least one solvent into the enclosure, wherein at least a portion of the solvent is in contact with the beads in the top internal cavity portion, to provide the reaction mixture in contact with the rotatable blades; iii. activating the heating assembly to bring the solvent within the enclosure to a temperature in the range of 40° C., to 100° C.; inserting at least one reactant into the reaction chamber within the enclosure, wherein the at least one reactant is in contact with the solvent and the beads in the top internal cavity portion, wherein the at least one reactant is in contact with the solvent and the beads in the top internal cavity portion; and inserting inert gas into the enclosure through the conduit; iv. spinning the impeller for a period of time in the range of 5 seconds to 90 seconds, at a rotational rate of at least 600 rounds per minute (rpm), to maintain the shear rate of at least 3·10³ sec⁻¹ within the reaction chamber, wherein the temperature within the reaction chamber is in the range of 40° C., to 100° C.; v. applying vacuum by the vacuum pump to the bottom internal cavity portion, wherein upon the application of vacuum the solvent is substantially evacuated through the conduit, and the beads are maintained in the top internal cavity portion by the semipermeable disc.

It is to be understood that the actions of step iii. above may be performed in any order, or simultaneously. Moreover, in a repetitive process according to the method of the present invention, steps iii, to iv (or (c) to (e) in various embodiments) are performed a number of cycles. In such cases, when the subsequent cycles are conducted, optionally the temperature and/or the inert gas atmosphere is already substantially adjusted from a previous cycle, when starting step (c). According to some embodiments, steps iii. and iv. are repeated a plurality of cycles, wherein the heating assembly remains activated throughout said cycled, such that a temperature in the range of 40° C., to 100° C. is maintained during said cycles.

According to some embodiments, inserting inert gas into the enclosure through the conduit entails switching the valve from a gas blocking state, in which the first distal conduit end is in fluid isolation from the bottom internal cavity portion to a gas transferring state, in which the first distal conduit end is in fluid communication with the bottom internal cavity portion.

According to some embodiments, applying vacuum by the vacuum pump to the bottom internal cavity portion entails operating the vacuum pump and switching the valve from a liquid blocking state, in which the second distal conduit end is in fluid isolation from the bottom internal cavity portion to a liquid transferring state, in which the second distal conduit end is in fluid communication with the bottom internal cavity portion.

According to some embodiments, the stirring apparatus is a mechanical stirrer and spinning of the impeller is performed at a rotational rate of 600 to 1000 rounds per minute, maintaining a shear rate of at least 3·10³ sec⁻¹.

According to other embodiments, spinning of the impeller is performed at a rotational rate of 5,000-30,000 rounds per minutes while maintaining a shear rate of at least 1·10⁶ sec⁻¹.

The term “solid phase synthesis” (SPS) means one or a series of chemical reactions used to prepare either a single compound or a library of molecularly diverse compounds, wherein the chemical reactions are performed on a compound that is bound to a solid phase support material through an appropriate linkage. Thus, according to the solid phase synthesis synthetic approach the compound or a precursor thereof is attached to a solid support during some or all of the synthetic steps. SPS is regularly implemented in the production of peptides.

According to some embodiments, the method comprises the steps:
(a) providing a reactor comprising a reaction chamber and a stirring apparatus comprising an impeller having at least two blades rotatable about an axis;
(b) inserting beads of functionalized polymeric resin and at least one solvent into the reactor to provide a reaction mixture, wherein the reaction mixture is in contact with the rotatable blades;
(c) inserting at least one protected monomeric organic molecule into the reaction chamber and spinning the impeller and at least one coupling agent, thereby forming a coupling product of the protected monomeric organic molecule and the resin;
(d) washing excess of said protected monomeric organic molecule; and
(c) inserting at least one deprotecting reagent into the reaction chamber and spinning the impeller, thereby removing at least one protecting group from the coupling product, forming a coupling product of a deprotected monomeric organic molecule and the resin, thereby completing a cycle in the solid phase synthesis of a peptide, a modified peptide or a hybrid thereof;
wherein the spinning of the impeller in at least one of steps (c) and (e) is performed at a temperature in the range of 40° C., to 100° C., for a period of time, at a rotational rate of at least 600 rounds per minute, while maintaining a shear rate of at least 3·10³ sec⁻¹, optionally wherein steps (c) to (e) are repeated a plurality of cycles.

According to some embodiments, there is provided a method for performing at least one cycle in the solid phase synthesis of a peptide, a modified peptide or a hybrid thereof, the method comprising the steps of:
(a) providing a reactor comprising a reaction chamber and a stirring apparatus, wherein the stirring apparatus comprises an impeller having at least two blades rotatable about an axis;
(b) inserting beads of functionalized polymeric resin and at least one solvent into the reactor to provide a reaction mixture, wherein the reaction mixture is in contact with the rotatable blades;
(c) inserting at least one protected monomeric organic molecule at least one coupling agent into the reaction chamber and spinning the impeller, thereby forming a coupling product of the protected monomeric organic molecule and the resin;
(d) washing excess of said protected monomeric organic molecule; and
(e) inserting at least one deprotecting reagent into the reaction chamber and spinning the impeller, thereby removing at least one protecting group from the coupling product, forming a coupling product of a deprotected monomeric organic molecule and the resin, thus completing a cycle in the solid phase synthesis of a peptide, a modified peptide or a hybrid thereof;
wherein the spinning of the impeller in at least one of steps (c) and (c) is performed at a temperature in the range of 40° C., to 100° C., for a period of time, at a rotational rate of at least 600 rounds per minute, while maintaining a shear rate of at least 3-103 sec⁻¹, optionally wherein steps (c) to (e) are repeated a plurality of cycles.

According to some embodiments, the spinning of the impeller in step (c) is performed at a temperature in the range of 40° C., to 100° C. According to some embodiments, the spinning of the impeller in step (e) is performed at a temperature in the range of 40° C., to 100° C. According to some embodiments, the spinning of the impeller in each one of steps (c) and (e) is performed at a temperature in the range of 40° C., to 100° C. According to some embodiments, the spinning of the impeller in step (c) is performed at a temperature in the range of 50° C., to 90° C. According to some embodiments, the spinning of the impeller in step (e) is performed at a temperature in the range of 50° C., to 90° C. According to some embodiments, the spinning of the impeller in each one of steps (c) and (e) is performed at a temperature in the range of 50° C., to 90° C. According to some embodiments, the spinning of the impeller in step (c) is performed at a temperature of at least 50° C., at least 55° C., at least 60° C., at least 65° C., at least 70° C., at least 75° C. or at least 80° C. Each possibility represents a separate embodiment of the invention. According to some embodiments, the spinning of the impeller in step (c) is performed at a temperature of no more than 105° C., no more than 100° C., no more than 90° C., or no more than 85° C. Each possibility represents a separate embodiment of the invention. According to some embodiments, the spinning of the impeller in step (e) is performed at a temperature of at least 50° C., at least 55° C., at least 60° C., at least 65° C., at least 70° C., at least 75° C. or at least 80° C. Each possibility represents a separate embodiment of the invention. According to some embodiments, the spinning of the impeller in step (e) is performed at a temperature of no more than 105° C., no more than 100° C., no more than 90° C., or no more than 85° C. Each possibility represents a separate embodiment of the invention. According to some embodiments, the spinning of the impeller in each one of steps (c) and (c) is performed at a temperature of at least 50° C., at least 55° C., at least 60° C., at least 65° C., at least 70° C., at least 75° C. or at least 80° C. Each possibility represents a separate embodiment of the invention. According to some embodiments, the spinning of the impeller in each one of steps (c) and (c) is performed at a temperature of no more than 105° C., no more than 100° C., no more than 90° C., or no more than 85° C. Each possibility represents a separate embodiment of the invention.

According to some embodiments, each one of steps (c) and (c) are performed sequentially at least 3 cycles, at least 3 cycles, at least 4 cycles, at least 5 cycles, at least 6 cycles, at least 7 cycles, at least 8 cycles, at least 9 cycles, or at least 10 cycles.

According to some embodiments, the method comprises the steps of:
i. providing the reactor as detailed herein;
ii, inserting beads of functionalized polymeric resin and at least one solvent into the enclosure to provide the reaction mixture in contact with the rotatable blades;
iii, inserting at least one protected monomeric organic molecule and at least one coupling agent into the reaction chamber within the enclosure;
iv(a) spinning the impeller for a period of time, at a rotational rate of at least 600 rounds per minute (rpm), to maintain the shear rate of at least 3·10³ sec⁻¹ within the reaction chamber, thereby forming a coupling product of the protected monomeric organic molecule and the resin;
iv(b), washing excess of said protected monomeric organic molecule; and
iv(c), inserting at least one deprotecting reagent into the reaction chamber within the enclosure;
iv(d). spinning the impeller for a period of time, at a rotational rate of at least 600 rounds per minute (rpm), to maintain the shear rate of at least 3-103 sec⁻¹ within the reaction chamber, thereby removing at least one protecting group from the coupling product, forming a coupling product of the deprotected monomeric organic molecule and the resin, thereby completing a cycle in the solid phase synthesis of a peptide, a modified peptide or a hybrid thereof;
wherein the method further comprises activating the heating assembly to bring the reaction mixture within the enclosure to a temperature in the range of 40° C., to 100° C. during step iv.

Optionally, the method comprises repeating steps iii. and iv(a)-(d) a plurality of times. For example, at least 2, at least 3, at least 4, at least 5, at least 6, at least 7, at least 8, at least 9 or at least 10 times. As detailed above a repetitive process according to the method of the present invention, steps iii, to iv. are performed a number of cycles. In such cases, when the subsequent cycles are conducted, optionally the temperature and/or the inert gas atmosphere is already substantially adjusted from a previous cycle, when starting step iii. According to some embodiments, steps iii, iv(a), iv(c) and iv(d) are repeated a plurality of cycles, wherein the heating assembly remains activated throughout said cycled, such that a temperature in the range of 40° C., to 100° C. is maintained during said cycles.

According to some embodiments, the method comprises the steps of:
i. providing the reactor as detailed herein;
ii, inserting beads of functionalized polymeric resin into the top internal cavity portion and inserting the at least one solvent into the enclosure, wherein at least a portion of the solvent is in contact with the beads in the top internal cavity portion, to provide the reaction mixture in contact with the rotatable blades;
iii. activating the heating assembly to bring the solvent within the enclosure to a temperature in the range of 40° C., to 100° C.;
inserting at least one protected monomeric organic molecule and at least one coupling agent into the reaction chamber within the enclosure, wherein each one of the at least one protected monomeric organic molecule and the at least one coupling agent is in contact with the solvent and the beads in the top internal cavity portion; and
inserting inert gas into the enclosure through the conduit;
iv(a) spinning the impeller for a period of time in the range of 5 seconds to 90 seconds, at a rotational rate of at least 600 rounds per minute (rpm), to maintain the shear rate of at least 3·10³ sec⁻¹ within the reaction chamber, thereby forming a coupling product of the protected monomeric organic molecule and the resin;
iv(b). applying vacuum by the vacuum pump to the bottom internal cavity portion, wherein upon the application of vacuum the solvent is substantially evacuated through the conduit, and the beads are maintained in the top internal cavity portion by the semipermeable disc; and
washing excess of said protected monomeric organic molecule; and
iv(c), inserting a solvent and at least one deprotecting reagent into the reaction chamber within the enclosure, wherein each one of the at least one deprotecting reagent and the inserted solvent is in contact the beads in the top internal cavity portion;
iv(d). spinning the impeller for a period of time in the range of 5 seconds to 90 seconds, at a rotational rate of at least 600 rounds per minute (rpm), to maintain the shear rate of at least 3×10³ sec⁻¹ within the reaction chamber, thereby removing at least one protecting group from the coupling product and forming a coupling product of the deprotected monomeric organic molecule and the resin, thereby completing a cycle in the solid phase synthesis of a peptide, a modified peptide or a hybrid thereof; and
v. applying vacuum by the vacuum pump to the bottom internal cavity portion, wherein upon the application of vacuum the solvent and excess deprotecting agent dissolved therein are substantially evacuated through the conduit, and the beads are maintained in the top internal cavity portion by the semipermeable disc;
wherein the method further comprises activating the heating assembly to bring the reaction mixture within the enclosure to a temperature in the range of 40° C., to 100° C. during step iv.

Optionally, the method comprises repeating steps iii. and iv(a)-(d) a plurality of times. For example, at least 2, at least 3, at least 4, at least 5, at least 6, at least 7, at least 8, at least 9 or at least 10 times.

According to some embodiments, the method further comprises a step of washing an excess of said deprotecting reagent, thereby isolating the coupling product of a deprotected monomeric organic molecule and the resin.

According to some embodiments, step (d) comprises washing excess of said protected monomeric organic molecule thereby separating the excess of said protected monomeric organic molecule from the coupling product of the protected monomeric organic molecule and the resin. According to some embodiments, step (d) further comprises discarding the separated excess of said protected monomeric organic molecule.

According to some embodiments, the method further comprises the steps of washing the reaction mixture and filtering the beads of polymeric resin by inserting a solvent into the top internal cavity portion and removing it from the bottom internal cavity portion, wherein the solvent is passed through the semipermeable disc and the beads are retained by the semipermeable disc in the top internal cavity portion.

According to some embodiments, step (d) comprises washing excess of said protected monomeric organic molecule thereby separating the excess of said protected monomeric organic molecule from the coupling product of the protected monomeric organic molecule and the resin in the reaction chamber. According to some embodiments, step (d) comprises washing excess of said protected monomeric organic molecule thereby separating the excess of said protected monomeric organic molecule from the coupling product of the protected monomeric organic molecule and the resin, such that the coupling product is remained in the reaction chamber.

According to some embodiments, each one of steps (c) and (e) are performed for a period of time, at a rotational rate of at least 600 rounds per minute, while maintaining a shear rate of at least 3·10³ sec⁻¹. According to some embodiments, each one of steps iii. and vi. are performed for a period of time, at a rotational rate of at least 600 rounds per minute, while maintaining a shear rate of at least 3-103 sec⁻¹.

According to some embodiments, the spinning in each one of steps (c) and (c) is performed of a period of time of no more than 2 minutes, no more than 1 minute, no more than 45 seconds or no more than 30 seconds. Each possibility represents a separate embodiment. According to some embodiments, the spinning in each one of steps iii. and iv. is performed of a period of time of no more than 2 minutes, no more than 1 minute, no more than 45 seconds or no more than 30 seconds. Each possibility represents a separate embodiment.

It is to be understood that the term “peptide”, as used herein refers to any one of “ordinary peptides”, which consist of a chain of amino acids; to similar chains, which include functional or unnatural amino acids instead or in addition to natural amino acids (e.g., peptidomimetics); and to modified peptides such as glycoproteins, proteoglycans and phosphopeptides, and peptide hybrids such as peptide-nucleic acids.

According to some embodiments, the method further comprises a step of cleaving the coupling product of step (e) from the resin thereby forming the peptide, thereby completing the solid phase synthesis thereof. According to some embodiments, the method further comprises a step of cleaving the coupling product of step iv. from the resin thereby forming the peptide, thereby completing the solid phase synthesis thereof. According to some embodiments, the method further comprises a step of cleaving the coupling product of step v. from the resin thereby forming the peptide, thereby completing the solid phase synthesis thereof. According to some embodiments, the method further comprises a step of cleavage of said peptide from said polymeric resin to provide the peptide in a cleaved form. According to some embodiments, the method further comprises a step of removal of side chain protecting group, to provide the peptide in an unprotected form.

Specifically, the skilled in the art of SPPS would appreciate that aside from the α-nitrogen, which is protected and deprotected to allow the sequential synthetic elongation of the peptide chain, other regions of the amino acids may be also protested by protected groups to allow the proper direction of elongation. Typically, the added amino acids include protected side chains, with protecting groups, which are removed under conditions, which are different from the conditions for removal of the α-nitrogen atom protecting group (i.e., the side chain protecting group will not be removed in conditions, which typically remove t-Boc or Fmoc). As the target peptide does not require the side-chain protection after the completion of the synthesis, the side chain protecting groups are typically removed at the step of cleaving the product from the solid phase support resin. The selection of protecting groups (e.g., side-chain protecting groups), which are retained during the solid phase synthesis under the conditions in which other protecting groups (e.g., the α-nitrogen protecting group) and are only removed at the end of, or later in, the synthesis in known as an orthogonal protecting group approach. According to some embodiments, the method further comprises a step of cleavage of said peptide, modified peptide or a hybrid thereof from said polymeric resin and removal of side chain protecting group, to provide the peptide, modified peptide or a hybrid thereof in a cleaved unprotected form.

According to some embodiments, the method further comprises a step of isolating the peptide from the reaction mixture.

It is to be understood that the term “monomeric organic molecule” refers to any organic molecule, which upon sequential or cyclical coupling to other organic molecules (e.g., in solid phase syntheses) will form a corresponding polymeric organic molecule. Typically, monomeric organic molecules in SPS may include natural or synthetic building blocks, such as amino acids or their analogs and/or saccharides or their analogs. However, the term “monomeric organic molecule”, as used herein further includes dimers, trimers and the like, which may act as building blocks in SPS. Thus, the methods provided herein include, for example, solid phase syntheses, where a resin bound to a monomeric amino acid is first coupled to a dipeptide, thereafter coupled to a tripeptide and then cleaved to form a hexapeptide.

According to some embodiments, the monomeric organic molecule is an amino acid or a saccharide. According to some embodiments, the monomeric organic molecule is an amino acid. According to some embodiments, the protected monomeric organic molecule is selected from the group consisting of an N-protected amino acid, an N-protected peptide. According to some embodiments, the monomeric organic molecule is a sugar molecule.

Thus, it is further to be understood that the term “polymeric organic molecule” refers to any organic molecule, which may be formed upon sequential or cyclical coupling to monomeric organic molecules (e.g., in solid phase syntheses). The nature of the polymeric organic molecule is dependent upon the identities of the monomeric organic molecules. Typically, polymeric organic molecules prepared in SPS may include natural/biological compounds, such as (poly)peptides, glycoproteins, proteoglycans and the like. As used herein, the term “polymeric organic molecule” refers to any molecule comprised of linked monomer units, as long as the molecule includes at least two chemically linked monomer units. The term “polymeric molecule” is intended to be inclusive of short oligomers, such as, but not limited to dimers, trimers and tetramers, as well as oligomers of about maximum 50 monomers and of long polymers including up to about one hundred monomer units. According to some embodiments the polymeric organic molecule comprises 2-50, 2-30, 3-25, 4-50, 4-100 or 5-120 monomeric units. Preferably, the polymeric molecule is a biological polymer, in particular a polypeptide or a hybrid thereof. According to some embodiments, the polymeric molecule is selected from the group consisting of: a peptide, a glycopeptide, a phospopeptide and a protcoglycan.

The term “cycle” as used herein refers to a sequence of steps, which may be repeated for a plurality of times. These steps are collectively referred to as a single cycle. Thus, “cyclical” refers to a method, which at least some of its steps repeat in a cyclical manner.

The term “plurality” as used herein refers to an integer, which is equal or higher than two.

According to some embodiments the at least one step of the solid phase synthesis is coupling of a monomer to one of: the polymeric resin; an oligomeric chain attached to the polymeric resin; and an additional monomer attached to the polymeric resin; wherein the monomer and additional monomer are each independently an amino acid; and wherein the oligomer is a peptide.

According to some embodiments the at least one step of the solid phase synthesis is coupling of an amino acid to the polymeric resin or to an amino acid or peptide chain attached to the polymeric resin.

According to some embodiments the at least one step of the solid phase synthesis comprises removal of a protecting group.

According to some embodiments the at least one step of the solid phase synthesis is selected from coupling of an amino acid to the resin, and removal of a protecting group. According to some embodiments the at least one step of the solid phase synthesis is selected from coupling of an amino acid to the resin, coupling of a saccharide to the resin, coupling of a hybrid amino acid to the resin, and removal of a protecting group. Each possibility represents a separate embodiment of the invention.

According to some embodiments the at least one step of the solid phase synthesis is selected from coupling of an amino acid to the resin and removal of a protecting group.

According to some embodiments the method comprises at least two steps of coupling a monomer to the resin and at least two steps of removal of a protecting group. According to some embodiments the method comprises at least two steps of coupling a monomer to the resin and at least two steps of removal of a protecting group, wherein the monomer is an amino acid or a saccharide. According to some embodiments the method comprises at least two steps of coupling a monomer to the resin and at least two steps of removal of a protecting group, wherein the monomer is an amino acid.

According to some embodiments the method comprises at least two steps of coupling of an amino acid to the resin and at least two steps of removal of a protecting group.

According to some embodiments the at least one step of the solid phase synthesis is cleaving the synthesized peptide, the modified peptide or the hybrid thereof from the solid resin.

The method of the present invention may be applied for synthesis of any peptide capable of being synthesized on a solid support. This includes standard peptides, as well as glycopeptides, phosphopeptides and proteoglycans.

According to some embodiments, the synthesis of the peptide, the modified peptide or the hybrid thereof includes multiple steps.

According to some embodiments, the solid phase synthesis method is for synthesis of polymeric organic molecule selected from the group consisting of: peptide, polypeptide, modified peptide, peptidomimetic, glycopeptide, phosphopeptide and proteoglycan. Each possibility represents a separate embodiment of the invention.

As used herein, the terms “polypeptide” and “oligopeptide” are well-known in the art, and are used to refer to a series of linked amino acid molecules. The terms are intended to include both short peptide sequences, such as, but not limited to a tripeptide, and longer protein sequences, optionally modified or hybridized.

In other embodiments, the solid phase synthesis method is for synthesis of a peptide, polypeptide, modified peptide, peptidomimetic, glycopeptide or proteoglycan. According to some embodiments, said organic molecule comprises a peptide chain.

According to some embodiments, the peptide is a non-modified peptide or non-hybridized peptide. According to some embodiments, the peptide is a non-modified or non-hybridized peptide. According to some embodiments, the modified peptide is a glycopeptide or a phosphopeptide.

It is to be understood that the terms “glycopeptides' and “glycoproteins” refer to peptides or proteins which contain carbohydrate units or oligosaccharide chains (glycans) covalently attached to amino acid side-chains.

It is to be further understood that the term “non-hybridized peptide” refers to peptides or proteins which contain only natural or unnatural, functionalized or non-functionalized amino acids, and does not include non-amino acid building blocks, such as saccharides.

According to some embodiments, the method of the present invention is used for synthesis of combinatorial libraries or arrays. According to some embodiments, combinatorial libraries or arrays of peptides are created using the methods of the present invention.

As used herein the term “amino acid” refers to an organic acid containing both a protected or unprotected amino group (NHPG or NH₂) and an acidic carboxyl group (COOH). Typically, amino acids include α-amino acids. These include, but are not limited to, the 25 amino acids that have been established as protein constituents. Amino acids contain at least one carboxyl group and one primary or secondary amino group on the amino acid molecule. Amino acids include such proteinogenic amino acids as alanine, valine, leucine, isoleucine, norleucine, proline, hydroxyproline, phenylalanine, tryptophan, methionine, glycine, serine, threonine, cysteine, cystine, tyrosine, asparagine, glutamine, aspartic acid, glutamic acid, lysine, hydroxylysine, ornithine, arginine, histidine, penicillamine and the like. The term “amino acid” is intended to include both unprotected amino acids and protected amino acids.

According to some embodiments, the amino acid comprises a protected amino acid. According to some embodiments, the amino acid comprises an N-protected amino acid.

The term “N-protected amino acid” as used herein refers to an amino acid in which the amino group (NH₂) is protected by an amino-protecting group and is thus protected from taking part in chemical reactions that can occur during the coupling reaction(s). As the most abundant amino acids in various fields of biology and medicine are α-amino acids, N-protected amino acids typically comprise amino-protecting groups covalently attached to the α-amines. However, the current invention further encompasses solid phase syntheses, which employ less frequently used building blocks, such as β-amino acids, where the amino group is separated from the carboxyl group by two carbon atoms. Thus, N-protected amino acids further comprise β-amino acids, where amino-protecting groups are covalently attached to the β-amines. According to some embodiments, the N-protected amino acid is selected from α-N-protected amino acid and β-N-protected amino acid. According to some embodiments, the N-protected amino acid is α-N-protected amino acid. It is to be understood that “α-N-protected amino acids” and “β-N-protected amino acids” respectively refer to α-amino acids comprising amino-protecting groups covalently attached to their α-nitrogen atom; and β-amino acids comprising amino-protecting groups covalently attached to their β-nitrogen atom.

The term “amino-protecting group” as used herein refers to a protecting group that preserves an amino group or an amino acid that otherwise would be modified by a chemical reaction in which an amino-containing compound (e.g., amino acid) is involved. Non-limiting examples of such protecting groups include the formyl group or lower alkanoyl group having from 2 to 4 carbon atoms, e.g., the acetyl or propionyl group; the trityl or substituted trityl groups, e.g., the monomethoxytrityl and dimethoxytrityl groups, such as 4,4′-dimethoxytrityl; the trichloroacetyl group; the trifluoroacetyl group; the silyl group; the phthalyl group; the (9-fluorenylmethoxycarbonyl) or “FMOC” group; the alkoxycarbonyl group, e.g., tertiary butoxy carbonyl (BOC); or other protecting groups derived from halocarbonates, such as, C6-Cn aryl lower alkyl carbonates. In the preparation of polypeptides in solid phase synthesis techniques, the FMOC group is typically employed.

As used herein the terms “sugar” and “saccharide” are interchangeable and refer to a compound comprising one or more monosaccharide groups. The term “monosaccharide” as used herein refers to the most basic units of carbohydrates. Monosaccharide are fundamental units of carbohydrates, which cannot be further hydrolyzed to simpler compounds. They are the simplest form of sugar and are usually colorless, water-soluble. and crystalline solids. Some monosaccharides have a sweet taste. Examples of monosaccharides include glucose, fructose and ribose. Monosaccharides are the building blocks of disaccharides (such as sucrose and lactose) and polysaccharides (such as cellulose and starch). With few exceptions (e.g., deoxyribose), monosaccharide have the chemical formula: Cₓ(H₂O)_{y}, where conventionally x>3. Monosaccharides can be classified by the number x of carbon atoms they contain: triose (3) tetrose (4), pentose (5), hexose (6), heptose (7), and so on. In aqueous solutions monosaccharides exist as rings if they have more than four carbons.

According to some embodiments, preferred monosaccharides are pentoses and/or hexoses. According to some embodiments, the monosaccharide comprises a protected monosaccharide. According to some embodiments, the monosaccharide comprises an O-protected monosaccharide.

The term “O-protected monosaccharide” as used herein refers to a monosaccharide in which one of its hydroxyl groups (OH) is protected by an oxygen-protecting group and is thus protected from taking part in chemical reactions that can occur during the coupling reaction(s). According to some embodiments the monosaccharide is selected from pentose and hexose. According to some embodiments the monosaccharide is a pentose. According to some embodiments the monosaccharide is a hexose. According to some embodiments the monosaccharide comprises a single free hydroxyl, wherein its remaining hydroxyl(s) comprise protecting group(s). According to some embodiments the monosaccharide is a hexose comprising four O-protected hydroxyl groups and one free hydroxyl group. According to some embodiments the monosaccharide is a pentose comprising three O-protected hydroxyl groups and one free hydroxyl group. The term “free” hydroxyl group, as used herein, refers to the unprotected OH chemical moiety. Similarly, the term “free” amine group, as used herein, refers to the unprotected NH₂ chemical moiety. Typically, “free” hydroxyl and/or amine groups are reactable in reactions, such as coupling reactions, whereas the corresponding protected groups would not undergo similar chemical reaction under similar conditions. According to some embodiments the method is for solid phase synthesis of an organic molecule comprising a total of 2-50, 2-20, 3-15, 3-10 or 3-6 residues selected from amino acid residues, nucleotide residues and saccharide residues. According to some embodiments the peptide comprises 2-50, 2-20, 3-15, 3-10 or 3-6 amino acid residues. According to some embodiments the glycoprotein comprises 2-50, 2-20, 3-15, 3-10 or 3-6 monosaccharide residues.

The term “oxygen-protecting group” as used herein refers to a protecting group that preserves an oxygen atom or a hydroxyl group that otherwise would be modified by a chemical reaction in which an oxygen-containing compound (e.g., tyrosine) is involved. Non-limiting examples of such protecting groups include the the trityl or substituted trityl groups, e.g., the monomethoxytrityl and dimethoxytrityl (DMT) groups, such as 4,4′-dimethoxytrityl; silyl ethers, such as trimethylsilyl and tert-butyldimethylsilyl; esters, such as acetate and halogenated acetates; lower alkyl groups, which may be substituted by a halogen atom or a cyano group; a benzyl group which may have a substituent; and a phenyl group which may have a substituent. In the preparation of polypeptides in solid phase synthesis techniques, trityl derivatives, in particular DMT are customarily employed.

The term “free” hydroxyl group, as used herein, refers to the unprotected OH chemical moiety. Similarly, the term “free” amine group, as used herein, refers to the unprotected NH₂ chemical moiety. Typically. “free” hydroxyl and/or amine groups are reactable in reactions, such as coupling reactions, whereas the corresponding protected groups would not undergo similar chemical reaction under similar conditions. According to some embodiments the method is for solid phase synthesis of an organic molecule comprising a total of 2-50, 2-20, 3-15, 3-10 or 3-6 amino acid residues. Each possibility represents a separate embodiment of the invention. According to some embodiments the peptide or a hybrid thereof comprises 2-50, 2-20, 3-15, 3-10 or 3-6 amino acid residues. Each possibility represents a separate embodiment of the invention. According to some embodiments the method is for solid phase synthesis of an organic molecule comprising a total of 2-50, 2-20, 3-15, 3-10 or 3-6 amino acid and/or saccharide residues. Each possibility represents a separate embodiment of the invention. According to some embodiments the peptide or a hybrid thereof comprises 2-50, 2-20, 3-15, 3-10 or 3-6 amino acid and/or saccharide residues. Each possibility represents a separate embodiment of the invention.

As used here in the terms “coupling”, “coupling process” or “coupling step” refer to a process of forming a bond between two or more molecules such as a two-monomer unit. A bond can be a covalent bond such as a peptide bond.

A peptide bond is a chemical bond formed between two molecules when the carboxyl group of one coupling molecule reacts with the amino group of the other coupling molecule, thereby releasing a molecule of water (H₂O). This is a dehydration synthesis reaction (also known as a condensation reaction), and usually occurs between amino acids. The resulting —C(═O)NH-bond is called a peptide bond, and the resulting molecule is an amide.

The terms “deprotection” and deprotecting as used herein refers to the removal of at least one protecting group. For example, deprotection comprises the removal of an amino-protecting group from a protected amino acid. More specifically, deprotection comprises replacing the FMOC protecting group attached to the amino group of a protected amino acid with a hydrogen atom, thereby forming a basic NH₂ group in a deprotected amino acid, according to some embodiments. The deprotection may be of one or a plurality of protecting groups. For a compound having n protecting groups, deprotecting will lead to the same compound having at least one less protecting group, i.e., the product compound will include between n−1 and 0 protecting groups.

The term “deprotected” refers to a compound, which underwent a removal of at least one protecting group. The term includes both compounds, which underwent removal of all of their protecting groups and compounds, which underwent removal of part of their protecting groups. For example, amino acids, such as lysine, arginine, aspartic acid, histidine, glutamic acid, serine, threonine, cysteine, tyrosine and the like, may include two protecting groups, a first protecting group covalently attached to the α-nitrogen and the second protecting group covalently attached to the reactive group of the side chain. In such cases, the deprotected amino acid may be defined as the amino acid after removal of only one protecting group or after removal of both protecting groups.

The term “unprotected” refers to a compound, which underwent a removal of at least one, or all of its protecting group(s), or did not include protecting groups from the beginning. In other words, unprotected compounds do not include protecting groups.

According to some embodiments, the method comprises the step of inserting at least two reactants into the reaction chamber.

According to some embodiments said at least one reactant is selected from the group consisting of: a deprotection agent, a coupling agent, and an amino acid. According to some embodiments said at least one reactant is selected from the group consisting of: a deprotection agent, a coupling agent, a saccharide and an amino acid. Each possibility represents a separate embodiment of the invention.

According to some embodiments said at least one reactant is selected from the group consisting of: a deprotection agent, a coupling agent, a saccharide, an amino acid or a modified or hybrid amino acid. According to some embodiments said at least one reactant is selected from the group consisting of: a deprotection agent, a coupling agent and an amino acid. According to some embodiments, said at least one reactant is selected from the group consisting of: a deprotection agent, a coupling agent and a protected monomeric organic molecule. According to some embodiments, said at least one reactant comprises a coupling agent and a protected monomeric organic molecule, thereby performing at least one coupling cycle of the solid phase synthesis of the organic molecule,

According to some embodiments, the protected monomeric organic molecule is an N-protected amino acid or an O-protected saccharide. According to some embodiments, the protected monomeric organic molecule is an O-protected saccharide. According to some embodiments said N-protected amino acid is an α-N-protected amino acid. According to some embodiments said N-protected amino acid comprises a protecting group covalently attached to its α-nitrogen. According to some embodiments said N-protected amino acid comprises a protecting group selected from an Fmoc protecting group and a tBoc protecting group. According to some embodiments said protecting group is selected from an Fmoc protecting group and a tBoc.

It is to be understood the protected N-protected amino acid may include other protecting group(s) at other regions of the molecule, as long as it contains the N-protection (e.g., the α-N-protection) or O-protection. Specifically, as detailed above, in any cases an orthogonal approach is required in selecting protecting group. For example, lysine is an amino acid having its α-nitrogen and an additional ε-nitrogen at its side chain group. The corresponding Fmoc-Lys(Boc)-OH is a protected lysine, in which its α-nitrogen is protected by Fmoc and its ε-nitrogen is protected by t-Boc. A solid phase synthesis may involve conditions, which orthogonally maintain the side-chain t-Boc, while removing the α Fmoc in some steps, and finally the t-Boc will also be removed. Thus, doubly protected amino acids, as Fmoc-Lys(Boc)-OH are encompassed under the term protected monomeric organic molecule, the term N-protected amino acid and the term α-N-protected amino acid.

According to some embodiments said N-protected amino acid is an Fmoc protected amino acid. According to some embodiments said protecting group is Fmoc protecting group.

According to some embodiments said N-protected amino acid comprises a Fmoc group covalently attached to its α-nitrogen

According to some embodiments, reactant comprises at least one deprotecting reagent.

According to some embodiments, the at least one deprotecting reagent is a reagent capable of removal of an Fmoc group. According to some embodiments said at least one reactant further comprises a reagent capable of removal of an Fmoc group.

According to some embodiments said reagent capable of removal of an Fmoc group comprises a base.

According to some embodiments said base comprises an amine.

According to some embodiments said amine is selected from the group consisting of piperidine, morpholine, piperazine, dicyclohexylamine, N,N-diisopropylethylamine, 4-dimethylaminopyridme, 1,8-diazabicycloundec-7-ene, pyrrolidme, cyclohexylamine, ethanolamine, diethylamme, trimethylamine, ammonia, tributylamine, 1,4-Diazabicyclo[2.2.2]octane, hydroxylamine, tris(2-aminoethyl)amine and combinations thereof.

According to some embodiments said at least one reactant further comprises a reagent capable of removal of an DMT group. Reagents capable of removal of an DMT group are generally acidic compounds, such as but not limited to dicholoacetic acid and/or trichloroacetic acid

According to some embodiments said reactants further comprise a coupling reagent. Specifically, carbodiimides are considered to be useful coupling reagents in the formation of amide bonds (e.g., peptide bonds), whereas various azole compounds are useful catalysts in formations of P—O bonds (e.g. phosphodiester bonds).

According to some embodiments, said coupling reagent comprises a combination of an amine and HATU (1-[Bis(dimethylamino)methylene]-1H-1,2,3-triazolo[4,5-b]pyridinium 3-oxide hexafluorophosphate).

According to some embodiments said coupling reagent comprises a carbodiimide.

According to some embodiments said inserting of at least one reactant to the reaction chamber comprises gradually inserting of at least one reactant to the reaction chamber. According to some embodiments the inserting of the carbodiimide to the reaction chamber comprises gradually inserting of the carbodiimide to the reaction chamber.

According to some embodiments said carbodiimide is selected from the group consisting of 1-ethyl-3-(3-dimethylaminopropyl)carbodiimide, N,N′-dicyclohexylcarbodiimide, N,N-Diisopropylcarbodiimide and combinations thereof.

According to some embodiments said carbodiimide comprises ethyl-3-(3-dimethylaminopropyl)carbodiimide.

According to some embodiments said reactants further comprise 1-hydroxybenzotriazole (HOBt).

According to some embodiments said coupling reagent comprises an azole catalyst. According to some embodiments said azole catalyst is selected from the group consisting of 1H-tetrazole, 2-ethylthiotetrazole, 2-benzylthiotetrazole, 4,5-dicyanoimidazole and combinations thereof.

According to some embodiments the method further comprises inserting at least one solvent into the reactor.

The term “solvent” as used herein refers to a fluid media, particularly liquid media, in which chemical transformation occur. The term is used broadly to include both liquid media. which dissolves the reactants involved in said transformation and media in which some of said reactants are insoluble. Thus, it is to be understood that in solid phase syntheses as described herein the reactant(s) may be soluble in the solvent, whereas the functionalized polymeric resin may be insoluble.

According to some embodiments the at least one solvent is selected the group consisting of from water, dimethylformamide, dichloromethane, N-methyl-2-pyrrolidone, dimethylacetamide and combinations thereof.

According to some embodiments said solvent comprises N-methyl-2-pyrrolidone.

According to some embodiments said solvent comprises water.

According to some embodiments said solvent comprises N-methyl-2-pyrrolidone and water.

According to some embodiments the method comprises an initial step of swelling said beads of polymeric resin in a solvent. According to some embodiments the step of swelling is performed after the step of inserting the beads and the solvent into the reactor; and prior to the step of inserting the reactant to the reaction chamber.

According to some embodiments the polymeric resin is in a concentration of 5-25% w/w, 5-20% w/w. 5-15% w/w or 8-12% w/w in the solvent.

According to some embodiments said swelling step comprises mixing said beads of polymeric resin for a specified period of time in said solvent.

According to some embodiments the swelling step is comprises mixing the beads of polymeric resin for a specified period of time in said solvent at a rotational rate in the range of 600-1400 rpm. According to some embodiments said rotational rate is in the range of 600-1200 rpm. According to some embodiments said rotational rate is in the range of 600-1000 rpm. According to some embodiments said rotational rate is in the range of 600-900 rpm. According to some embodiments said rotational rate is in the range of 600-800 rpm. According to some embodiments said rotational rate is in the range of 600-700 rpm.

According to some embodiments the swelling step is comprises mixing the beads of polymeric resin for a specified period of time in said solvent at a temperature in the range of 40-100° C. According to some embodiments, the temperature is in the range of 45-90° C. or 50-80° C.

According to some embodiments said period of time is in the range of 10-150, 20-120 or about 30 seconds.

According to some embodiments said mixing said beads of polymeric resin for a specified period of time in said solvent, comprises maintaining shear stress of at least 1.5 N/m². According to some embodiments said shear stress is in the range of 1.5-5, 1.8-3.8, 1.8-3.0 or 1.8-2.4 N/m².

In other embodiments, a shear stress of 500-2000 N/m² is maintained. According to some embodiments, a shear stress of 750-1500, or 900-1200 N/m² is maintained.

A functionalized polymeric resin according to the invention is any polymeric resin comprising a reactive group to which an organic molecule (e.g., an amino acid or a peptide) may be coupled.

A reactive group of a polymeric resin according to the invention includes but is not limited to, an amino group, hydroxyl group carboxy group, a carboxylic derivative group, such as acyl halide, halo group and pseudo-halo group, such as a sulfonate derivative.

According to some embodiments, the functionalized resin comprises a residue is an amino acid residue. According to some embodiments, the functionalized resin comprises an amino acid residue.

According to some embodiments said functionalized beads of polymeric resin comprises polystyrene-divinylbenzene.

According to some embodiments said polystyrene-divinylbenzene comprising resin is selected from 1% DVB-PS chloromethylated resin and Rink Amide Tentagel resin.

According to some embodiments said resin is 1% DVB-PS-chloromethylated resin.

According to some embodiments said resin is Rink Amide Tentagel resin.

According to some embodiments said beads of polymeric resin have coupling capacity in the range of 0.2-1.0 mmol/g, 0.3-0.9 mmol/g or 0.35-0.8 mmol/g. Advantageously, the beads of polymeric resin have coupling capacity higher than 1.0 milliequivalents/gram, which reduces the total cost of the reaction starting materials. According to some embodiments, the beads of polymeric resin have coupling capacity in the range of 1.0-3.0 mmol/g. According to some embodiments, the beads of polymeric resin have coupling capacity in the range of 1.0-2.0 mmol/g, 1.5-2.5 mmol/g or 2.0-3.0 mmol/g.

According to some embodiments said beads of polymeric resin have particle size in the range of 20-200, 50-180, 60-160, 65-85, 70-40 or 80-120 μm.

According to some embodiments said mechanical stirrer comprises at least three blades rotatable about an axis. According to some embodiments said mechanical stirrer comprises three blades rotatable about an axis. According to some embodiments said blades rotatable about an axis are spinning upwards at a first angle. According to some embodiments said first angle is in the range of 20-40°, 25-35° or 30-35°.

According to some embodiments said blades rotatable about an axis are spinning downwards at a second angle. According to some embodiments said second angle is in the range of 30-50°. 35-45° or 40-45°.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIG. 1A is a schematic illustration of an Extremely Fast SPS (HF-SPS) apparatus configured to enable high shear rate stirring at elevated temperature, according to some embodiments.

FIG. 1B is a schematic illustration of an Extremely Fast SPS (HF-SPS) apparatus configured to enable high shear rate stirring at elevated temperature, according to some embodiments.

FIG. 1C is a schematic illustration of an Extremely Fast SPS (HF-SPS) apparatus configured to enable high shear rate stirring at elevated temperature, according to some embodiments.

FIG. 2 is a general scheme of the present method to prepare somatostatin 14, according to some embodiments.

FIG. 3 is an HPLC chromatogram of a somatostatin 14 produced by the method of the present invention, according to some embodiments.

FIG. 4 is a close-up photograph of the resin beads at the end of the method of the present invention, according to some embodiments.

FIGS. 5A-E describe procedures and results relating to the preparation of Peptide-a (KLLQDILDA) through Route 1: Sequence with protecting groups during peptide assembly (FIG. 5A); procedure data (FIG. 5B); Microscope image of beads after synthesis (FIG. 5C); Analytical HPLC of the crude (220 nm) (FIG. 5D); and MS data (FIG. 5E).

FIGS. 6A-E describe procedures and results relating to the preparation of Peptide-a (KLLQDILDA) through Route 2: Sequence with protecting groups during peptide assembly (FIG. 6A); procedure data (FIG. 6B); Microscope image of beads after synthesis (FIG. 6C); Analytical HPLC of the crude (220 nm) (FIG. 6D); and MS data (FIG. 6E).

FIGS. 7A-E describe procedures and results relating to the preparation of Peptide-a (KLLQDILDA) through Route 3: Sequence with protecting groups during peptide assembly (FIG. 7A); procedure data (FIG. 7B); Microscope image of beads after synthesis (FIG. 7C); Analytical HPLC of the crude (220 nm) (FIG. 7D); and MS data (FIG. 7E).

FIGS. 8A-E describe procedures and results relating to the preparation of the peptide HFGWI through Route 3: Sequence with protecting groups during peptide assembly (FIG. 8A); procedure data (FIG. 8B); Microscope image of beads after synthesis (FIG. 8C); Analytical HPLC of the crude (220 nm) (FIG. 8D); and MS data (FIG. 8E).

FIGS. 9A-E describe procedures and results relating to the preparation of the peptide HFG through Route 3: Sequence with protecting groups during peptide assembly (FIG. 9A); procedure data (FIG. 9B); Microscope image of beads after synthesis (FIG. 9C); Analytical HPLC of the crude (220 nm) (FIG. 9D); and MS data (FIG. 9E).

FIGS. 10A-E describe procedures and results relating to the preparation of the peptide (D)HFG through Route 3: Sequence with protecting groups during peptide assembly (FIG. 10A); procedure data (FIG. 10B); Microscope image of beads after synthesis (FIG. 10C); Analytical HPLC of the crude (220 nm) (FIG. 10D); and MS data (FIG. 10E).

FIG. 11 is an analytical HPLC recorder for co-injection of (D)HFG and HFG.

FIGS. 12A-E describe procedures and results relating to the preparation of VAS ((L)CYFQN(L)CPRG) through Route 3: Sequence with protecting groups during peptide assembly (FIG. 12A); procedure data (FIG. 12B); Microscope image of beads after synthesis (FIG. 12C); Analytical HPLC of the crude (220 nm) (FIG. 12D); and MS data (FIG. 12E).

FIGS. 13A-E describe procedures and results relating to the preparation of DDVAS ((D)CYFQN(D)CPRG) through Route 3: Sequence with protecting groups during peptide assembly (FIG. 13A); procedure data (FIG. 13B); Microscope image of beads after synthesis (FIG. 13C); Analytical HPLC of the crude (220 nm) (FIG. 13D); and MS data (FIG. 13E).

FIGS. 14A-C summarize conditions and results for preparing SOM (AGCKNFFWKTFTSC) though various routes. FIG. 14A summarizes conditions for preparing SOM via Routes 3, 6 and MW, and analytical HPLC chromatogram of SOM synthesis. FIG. 14B summarizes results and conditions for preparing SOM via Routes 2-6 and MW. FIG. 14C is a bar chart histogram summarizing purities achieved by preparing SOM via Routes 2-6 and MW.

FIGS. 15A-D describe procedures and results relating to the preparation of SOM (AGCKNFFWKTFTSC) through Route 2: Sequence with protecting groups during peptide assembly (FIG. 15A); procedure data (FIG. 15B); Microscope image of beads after synthesis (FIG. 15C); and analytical HPLC of the crude (220 nm) (FIG. 15D).

FIGS. 16A-E describe procedures and results relating to the preparation of SOM (AGCKNFFWKTFTSC) through Route 3: Sequence with protecting groups during peptide assembly (FIG. 16A); procedure data (FIG. 16B); Microscope image of beads after synthesis (FIG. 16C); analytical HPLC of the crude (220 nm) (FIG. 16D); and MS data (FIG. 16E).

FIGS. 17A-E describe procedures and results relating to the preparation of SOM (AGCKNFFWKTFTSC) through Route 4: Sequence with protecting groups during peptide assembly (FIG. 17A); procedure data (FIG. 17B); Microscope image of beads after synthesis (FIG. 17C); analytical HPLC of the crude (220 nm) (FIG. 17D); and MS data (FIG. 17E).

FIGS. 18A-E describe procedures and results relating to the preparation of SOM (AGCKNFFWKTFTSC) through Route 5: Sequence with protecting groups during peptide assembly (FIG. 18A); procedure data (FIG. 18B); Microscope image of beads after synthesis (FIG. 18C); analytical HPLC of the crude (220 nm) (FIG. 18D); and MS data (FIG. 18E).

FIGS. 19A-E describe procedures and results relating to the preparation of SOM (AGCKNFFWKTFTSC) through Route 6: Sequence with protecting groups during peptide assembly (FIG. 19A); procedure data (FIG. 19B); Microscope image of beads after synthesis (FIG. 19C); analytical HPLC of the crude (220 nm) (FIG. 19D); and MS data (FIG. 19E).

FIGS. 20A-E describe procedures and results relating to the preparation of SOM (AGCKNFFWKTFTSC) through microwave: Sequence with protecting groups during peptide assembly (FIG. 20A); procedure data (FIG. 20B); Microscope image of beads after synthesis (FIG. 20C); analytical HPLC of the crude (220 nm) (FIG. 20D); and MS data (FIG. 20E).

FIGS. 21A-E describe procedures and results relating to the preparation of MARADONA through Route 6: Sequence with protecting groups during peptide assembly (FIG. 21A); procedure data (FIG. 21B); Microscope image of beads after synthesis (FIG. 21C); analytical HPLC of the crude (220 nm) (FIG. 21D); and MS data (FIG. 21E).

### DETAILED DESCRIPTION OF THE INVENTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

The methods of the present invention are suitable of synthesis of any peptide which can be synthesized on a solid support. The methods are particularly suitable for peptides produced using multiple synthesis steps and requiring orthogonal protection of reactive groups.

The methods of the present invention provide at least one improvement in synthesis parameters, including but not limited to synthesis time, synthesis yield, and reduction of side product formation.

Without wishing to be bound to any mechanism of action, these improvements may be due to applying high shear force during synthesis steps, elimination of accumulation of reactants or intermediates in the reaction mixture, and maintaining of reaction mixtures having improved homogeneity.

Basic understanding of hydrodynamic energies, as well as shear and compressive stress and forces applied to the reaction media could be critical to the success of scale up synthesis processes. Nevertheless, up till now very few investigations were made on the influence of the hydrodynamic parameters on the yield and side reactions in SPS. Importantly, the influence of a combination of high shear mixing properties and elevated reaction temperatures was not investigated thus far as it was believed that it may be detrimental to the structural integrity of polymeric resin beads, which are essential in solid phase syntheses. Some important hydrodynamic parameters include ‘shear rate’, ‘shear stress’, and ‘shear force’.

The term ‘shear rate’, measured in inverse seconds (SI unit) refers to rate at which a progressive shearing deformation is applied to a material. As used herein, shear rate refers to the rate at which the deformation is applied to the polymeric resin beads within the reaction mixture while being mixed. Generally, the shear rate for a fluid flowing between two parallel plates, one moving at a constant speed and the other one stationary is defined by:

\dot{γ}=v/h′
wherein γ is the shear rate; v is the velocity of the moving plate (in sec⁻¹); and h is the distance between the two parallel plates. For the simple shear case, it is just a gradient of velocity in a flowing material.

In stirred tanks, the following correlations were derived [Perez et al. Chem Eng. J. 124, 2006, 1; and Metzner and Otto AIChE Journal, 3, 1957, 3]:

$$γ = \frac{P}{τ \cdot V} γ = {k}_{i} N$$

wherein kᵢ is an impeller constant; N is the agitation speed, (i.e. the rotational speed of the impeller) measured in sec⁻¹; τ is the shear stress measured in pascal (newton per square meter); P is the power input (in Watts), which depends on the torque of the impeller and on its rotational speed (N); and V is the volume of the fluid in the tank.

The term ‘shear stress’, measured in inverse seconds refers to a component of stress coplanar with a material cross section. As used herein, shear stress refers to the component of stress applied on the polymeric beads, which is coplanar with their cross section. Generally, shear stress arises from the force vector component parallel to the cross section.

Being a measure of stress, shear stress is measured in force per unit area (in SI units: N/m²)

τ=F/A′
wherein τ is the shear stress; F is the force applied; and A is the cross-sectional area of material with area parallel to the applied force vector (i.e. cross-sectional area of the beads).

With reference to other hydrodynamic parameters, shear stress can also be derived from shear rate by: τ=\dot{γ}μ where μ is the dynamic viscosity of the fluid.

The term ‘viscosity’ refers to a hydrodynamic property of a fluid depicting its measure of resistance to gradual deformation by shear stress or tensile stress. Viscosity arises from collisions between neighboring particles in the fluid that are moving at different velocities. As used herein, viscosity refers to the viscosity of the reaction mixture comprising the solvent, the beads and other added reagents.

The term ‘shear force’ refers to a force acting in a direction parallel to a surface or to a planar cross section of a body. As used herein, shear force refers to the force acting in a direction parallel to a surface or to a planar cross section of the polymeric beads while being mixed. Shear force, Fs, can be derived from shear stress as it consists of the integrated shear stress (τ) over the surface area (A) of a body.

Fs=∫_{A}τdA

Being heterogeneous reactions, (i.e., where the protected amino acids are in solution, whereas the resin is not) the reactions performed in the SPS cycle are diffusion-controlled reactions highly affected by stirring or agitation. As described above gentle mixing methods are routinely employed in the large-scale production of peptides (e.g. vortex, nitrogen stream, rotary evaporator rotor, agitation by rocking), such that a low shear stress over the polymeric beads is maintained thereby avoiding damage thereof. In contrast with the gentle mixing approach in solid phase synthesis, mixing methods used in the production of small molecules in solution are frequently performed in high rpm.

The influence of the typically employed gentle mixing in SPS is not well documented and characterized. There are two main parameters that could be critical in the steps of the SPS cycle (i.e., coupling of protected building block; deprotection and washing). First, compressive and shear stress applied to the resin beads could cause them to break and as a consequence the isolation process will be tedious due to slow filtration and possible blockage of the filter. Second, the rates of the coupling and the deprotection reactions and the washing steps might be influenced by breaking up of the beads. Moreover, a secondary parameter stem from the interaction between the solid phase (resin beads) and the liquid phase. The better distribution of the resin beads, caused by circular flow rate of the beads in the media, could increase the mass transfer between the liquid bulk to the solid surface and thus accelerate the reaction. In specific reactions, it could decrease the generation of impurities and improve the impurity profile of the final product.

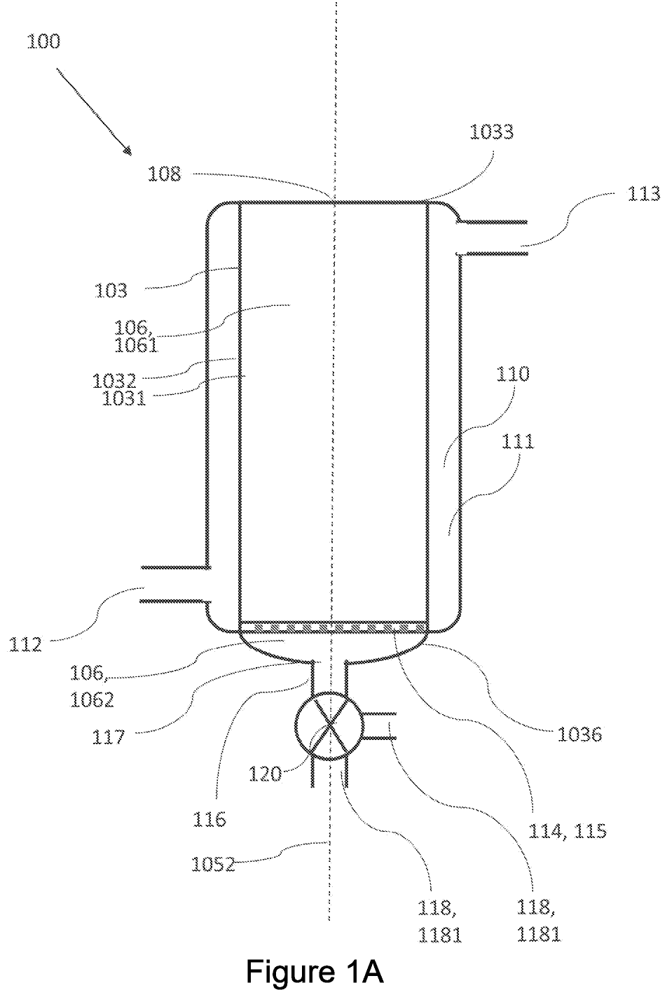

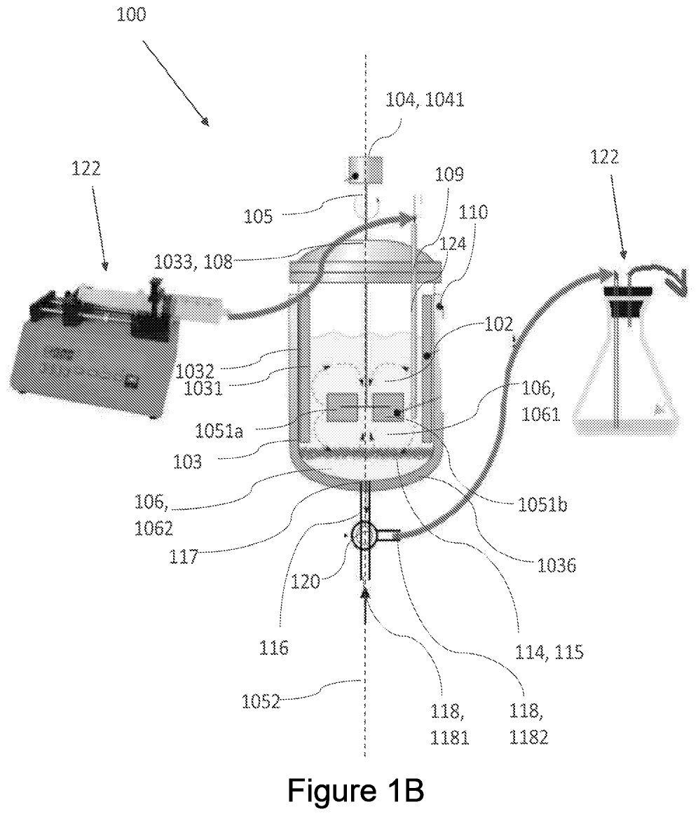

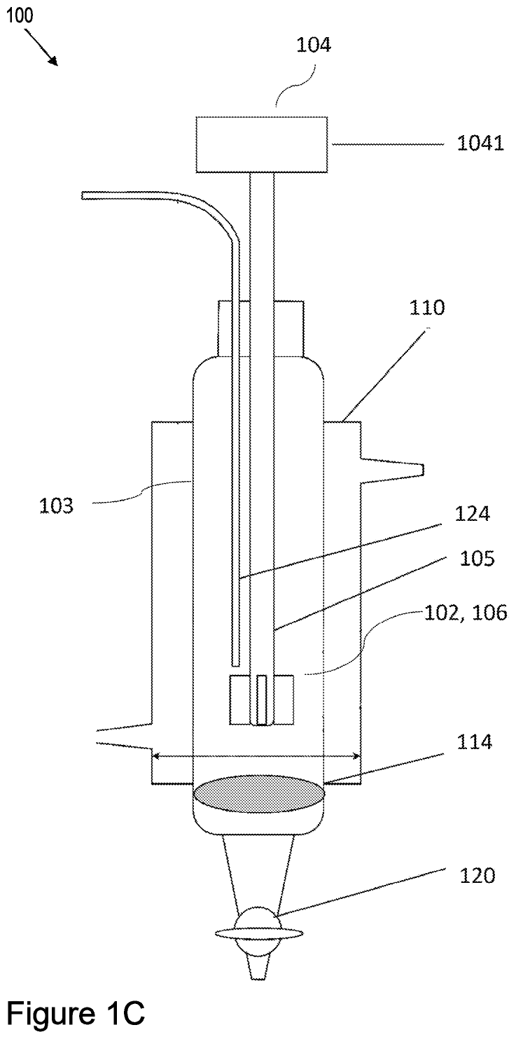

Reference is now made to FIGS. 1A-1C, which are schematic representations of chemical reactor apparatus according to the present invention. According to some embodiments, the method of the present invention is carried out in a reactor as presented in FIG. 1A, FIG. 1B or FIG. 1C. According to some embodiments, any one of the method steps of any one of the methods described above, is carried out in an apparatus as presented in FIG. 1A, FIG. 1B or FIG. 1C. As the apparatuses depicted in FIG. 1A-C are similar, they will be mostly addressed collectively. Distinctions will be referred separately and can be appreciated by the skilled in the art.

According to some embodiments, there is provided a reactor 100. Reactor 100 comprises a reaction chamber 102 and a stirring apparatus 104. Stirring apparatus 104 and its various components are shown in FIGS. 1B-C, however similar components may be used for the reactor 100 of FIG. 1A. Specifically, stirring apparatus 104 comprises an impeller 105 having at least two blades 1051 rotatable about an axis 1052. FIG. 1B depicts an impeller 105 having at least two blades 1051, of which blade 1051a and blade 1051b are pointed out. FIG. 1C depicts a five-fin turbine agitator impeller.

A stirring apparatus 104 according to the invention is a stirrer, such as a mechanical stirrer or a homogenizer-type stirrer, comprising at least two rotatable blades 1051 and operated by a stirring apparatus motor 1041. Any stirrer that is capable to being used to stir a solid phase synthesis reaction and create rotational rate and shear rate indicated herein, may be used of according to the present invention.

According to some embodiments, the reactor 100 further comprises an enclosure 103, which defines an internal cavity 106 and comprises the reaction chamber 102. According to some embodiments, the enclosure has an enclosure internal face 1031 facing the internal cavity 106 and an enclosure external face 1032.

According to some embodiments, each one of the at least two blades 1051 is disposed within the internal cavity 106

According to some embodiments, the enclosure 103 is substantially cylindrical

According to some embodiments, the stirring apparatus 104 and the enclosure 103 are substantially coaxial. According to some embodiments, the stirring apparatus 104 and the reaction chamber 102 are substantially coaxial. According to some embodiments, the impeller 105 and the enclosure 103 are substantially coaxial. According to some embodiments, the impeller 105 and the reaction chamber 102 are substantially coaxial. According to some embodiments, the coaxial elements described in the present paragraph are coaxial along axis 1052.

According to some embodiments, any of the stirring apparatuses 104 disclosed herein, such as mechanical stirrers, comprise a generally elongated body, which align along axis 1052.

Similarly, according to some embodiments, the enclosure 103 has a generally elongated body. Typical relative dimensions between the length of the enclosure 103 (i.e., the distance along the axis of the stirring apparatuses 104) to the width of the enclosure 103 (i.e., the distance perpendicular to the axis of the stirring apparatuses 104) are about 6:1. According to some embodiments, the length to width ration of the enclosure 103 is in the range of 15:1 to 2:1, 12:1 to 3:1; 10:1 to 4:1, 9:1 to 9:2 or 8:1 to 5:1. Each possibility represents a separate embodiment of the invention.

The enclosure 103 of the present reactor 100, according to some embodiments, is three dimensional and includes at least one first opening 108 for the insertion of the stirring apparatus 104. According to some embodiments, the opening 108 is located substantially in the center of a top enclosure face 1033. In such manner, enclosure 103 and the impeller 105 are substantially coaxial, according to some embodiments.

According to some embodiments, the stirring apparatus 104 is configured to mix a liquid mixture at a rotational rate of at least 600 rounds per minute. According to some embodiments, the stirring apparatus 104 is configured to mix a liquid mixture at a rotational rate of at least 600 rounds per minute, while maintaining a shear rate of at least 3·10³ sec⁻¹ thereby performing at least one step of the solid phase synthesis.

According to some embodiments, the stirring apparatus 104 is a mechanical stirrer configured to spin the at least two blades 1051 at a rotational rate of 600 to 1400 rounds per minute. According to some embodiments, the stirring apparatus 104 is a mechanical stirrer and spinning of the impeller 105 is performed at a rotational rate of 600 to 1000 rounds per minute, maintaining a shear rate of at least 3·10³ sec⁻¹. According to other embodiments, spinning of the impeller 105 is performed at a rotational rate of 5,000-30,000 rounds per minutes while maintaining a shear rate of at least 1·10⁶ sec⁻¹.

According to some embodiments, the reactor 108 further comprises a heating assembly 110.

According to some embodiments, the heating assembly 110 is configured to elevate the temperature with the reaction chamber 102 to a temperature in the range of 40° C., to 100° C. According to some embodiments, the heating assembly 110 is configured to elevate the temperature with the enclosure 103 to a temperature in the range of 40° C., to 100° C. According to some embodiments, the temperature is in the range of 50° C., to 90° C. According to some embodiments, the temperature is at least 30° C., at least 35° C., at least 40° C., at least 45° C., at least 50° C., at least 55° C., at least 60° C., at least 65° C., at least 70° C., or at least 75° C. Each possibility represents a separate embodiment.

According to some embodiments the heating assembly 110 is in contact with the external enclosure face 1032.

According to some embodiments, the heating assembly 110 is surrounding the enclosure 103. According to some embodiments, the enclosure 103 is substantially cylindrical and is surrounded by the heating assembly 110.

According to some embodiments, the heating assembly 110 is selected from a circulating fluid bath and a heating jacket.

Specifically, FIG. 1A is showing a circulating fluid bath assembly 110. Circulating fluid bath assembly 110 comprises a fluid bath 111 having a heating fluid inlet 112 and a heating fluid outlet 113. Typically, during the operation of circulating fluid bath assemblies, such as circulating fluid bath assembly 110, a heating fluid, such as water or an oil, is heated externally by a heater and circulated when at the desired temperature within fluid bath 111 from heating fluid inlet 112 to heating fluid outlet 113. After exiting heating fluid outlet 113, the heating fluid comes again in contact with the heater to adjust its temperature and re-enters fluid bath 111 from heating fluid inlet 112. It is to be understood that when the heating fluid is within fluid bath 111. it is in contact with enclosure external face 1032, such that enclosure 103 is being heated thereby.

FIG. 1B is showing a heating jacket 110. Heating jacket 110 is a cylindrical jacket adapted to be placed around an article to be heated. Thus, heating jacket 110 is a cylindrical jacket adapted to be placed around enclosure 103. Heating jacket 110 includes a flexible heater, such as a flexible heating coil, and is disposed over enclosure 103 so that, when activated, the flexible heater elevates the temperature of the enclosure 103 and thus the contents located therein. The flexible heater includes, according to some embodiments, flexible heating element enclosed in multiple layers of flexible high temperature resistant cloth configured for electric insulation. The heating jacket 110 can also include a thermostat enclosed within the layers to regulate the temperature of the heating element.

According to some embodiments, the heating assembly 110 is a heating jacket. According to some embodiments, the heating assembly 110 is a circulating fluid bath. According to some embodiments, the heating assembly 110 is configured to transfer heat to the external enclosure face 1032 from a heated fluid circulating in the heating assembly 110. According to some embodiments, the heating assembly 110 is configured to transfer heat to the external enclosure face 1032 from a heating coil within the heating assembly 110. According to some embodiments, the heating assembly 110 is not a microwave heater.

According to some embodiments, the internal cavity defined 106 by the enclosure 103 is divided by a top internal cavity portion 1061 and a bottom internal cavity portion 1062 by a semipermeable disc 114 having a plurality of pores 115 therein. According to some embodiments, the internal cavity 106 is divided by a semipermeable disc 114 to a top internal cavity portion 1061 and a bottom internal cavity portion 1062, wherein the semipermeable disc 114 is having a plurality of pores 115 therein. According to some embodiments, the top internal cavity portion 1061 has a greater volume than the bottom internal cavity portion 1062. According to some embodiments, the semipermeable disc 114 is in contact with the internal enclosure face 1031. According to some embodiments, the semipermeable disc 114 is flat and round, and the enclosure 103 has a matching round cross section, wherein the semipermeable disc 114 is in contact with the internal enclosure face 1031 throughout said cross-section.

According to some embodiments, the semipermeable disc 114 is permeable to fluids. According to some embodiments, the semipermeable disc 114 is impermeable to solids above a specified diameter. According to some embodiments, the semipermeable disc 114 is impermeable to the beads of the functionalized polymeric resin of the present method. According to some embodiments, the semipermeable disc 114 is permeable to fluids and impermeable to the beads of the functionalized polymeric resin. According to some embodiments, the semipermeable disc 114 is positioned within the enclosure 103 such that fluid communication is enabled between the top internal cavity portion 1061 and the bottom internal cavity portion 1062. and passage of the polymeric resin beads is prevented between the top internal cavity portion 1061 and the bottom internal cavity portion 1062. Thus, according to some embodiments, the process of the present invention comprises placing the polymeric resin beads on the semipermeable disc, at detailed above, such that they are within the top internal cavity portion.

According to some embodiments, the rotatable blades 1051 are disposed within the top internal cavity portion 1061.

Therefore, with respect to the method of the present invention, according to some embodiments, the polymeric resin beads are within the top internal cavity portion 1061 during step iv. According to some embodiments, upon inserting the beads of the functionalized polymeric resin in step ii., the beads are confined within the top internal cavity portion 1061. According to some embodiments, at least a portion of the solvent is within the top internal cavity portion 1061 during step iv. specifically. FIG. 1B shows the solvent (not numbered), in a situation in which a majority thereof resides in the top internal cavity portion 1061 and a minority thereof resides inside the bottom internal cavity portion 1062. According to some embodiments, the reaction chamber 102 is within the top internal cavity portion 1061. According to some embodiments, in step iv, the shear rate and the temperature range are maintained within the top internal cavity portion 1061. According to some embodiments, the reaction chamber 102 is within the top internal cavity portion 1061, and wherein in step iv, the shear properties and the temperature range are maintained there within.

According to some embodiments, the semipermeable disc 114 is a glass fritted disc.

According to some embodiments, the fritted disc has nominal maximum pore size of at least 40 micron. According to some embodiments, the fritted disc has nominal maximum pore size in the range of 40 microns to 100 microns.

According to some embodiments, the reactor further comprises a conduit 116 extending from a proximal conduit end 117 to a distal conduit end 118, wherein the proximal conduit end 117 is connected to a bottom portion of the enclosure 1036, which encloses the bottom internal cavity portion 1062. According to some embodiments, each one of the proximal conduit end 117 and the distal conduit end 118 is an open end. According to some embodiments, the distal conduit end 118 is located out of the enclosure 103. According to some embodiments, the reactor 100 further comprises a valve 120 configured to monitor flow of fluids within the conduit 116. According to some embodiments, when the valve 120 is in an open state, fluid communication between the bottom internal cavity portion bottom internal cavity portion 1062 and the distal conduit end 118 is enabled. According to some embodiments, when the valve 120 is in a closed state, fluid communication between the bottom internal cavity portion 1062 and the distal conduit end 118 is prevented.

According to some embodiments, the distal conduit end 118 is directly or indirectly connected to an inert gas cylinder (not shown in the figures), or a vacuum pump (not shown in the figures). According to some embodiments, the distal conduit end 118 is directly or indirectly connected to at least one of an inert gas cylinder and a vacuum pump. According to some embodiments, the distal conduit end 118 is directly or indirectly connected to an inert gas cylinder. According to some embodiments, the distal conduit end 118 is directly or indirectly connected to a vacuum pump. According to some embodiments, the valve 120 is configured to monitor flow of inert gasses and/or liquids. According to some embodiments, the conduit 116 is configured to enable flow of inert gasses and/or liquids.

It is to be understood that specific valves and conduits are made of materials, which enable flow of gasses or liquids based on the requirement. For example, gas valves and conduits are to be able to withstand high gas pressures and valves and conduits in chemical reactor are made of inert material, which do not substantially degrade upon prolonged contact with chemical reagents and solvents at elevated temperatures.

According to some embodiments, the distal conduit end 118 is directly or indirectly connected to an inert gas cylinder, wherein the valve 120 is configured to monitor flow of inert gasses, and/or wherein distal conduit end 118 is connected directly or indirectly to a vacuum pump, wherein the valve 120 is configured to monitor flow of liquids.

According to some embodiments, the reactor comprises a three-way bidirectional conduit 116 extending from a proximal conduit end 117 to a first distal conduit end 1181 and to a second distal conduit end 1182. Three-way conduits 116 are shown in FIGS. 1A and 1B. However, the present invention is no limited to such valve and a two-way valve is depicted in FIG. 1C. According to some embodiments, conduit 116 enables flow of fluids therethrough in both directions, i.e., from the proximal conduit end 117 to the distal conduit end 118 and vice versa.

According to some embodiments, the conduit 116 comprises at least three ends.

According to some embodiments, each of the conduit ends (117, 118, 1181, 1182) is an open end, such that fluid communication may be enabled between the proximal conduit end 117 and each of the distal conduit ends (118, 1181, 1182).

According to some embodiments, the proximal conduit end 117 is connected to a bottom enclosure portion 1036, wherein bottom enclosure portion 1036 encloses the bottom internal cavity portion 1062. According to some embodiments, the first distal conduit end 1181 is connected directly or indirectly to an inert gas source. According to some embodiments, the second distal conduit end is connected directly or indirectly to a vacuum pump 1182. According to some embodiments, the reactor 100 further comprises a three-way valve 120 configured to monitor flow of both liquids and gasses through the conduit 116.

According to some embodiments, the reactor 100 further comprises a three way bidirectional conduit 116 extending from a proximal conduit end 117 to a first distal conduit end 1181 and to a second distal conduit end 1182, wherein the proximal conduit end 117 is connected to a bottom portion of the enclosure 1036, which encloses the bottom internal cavity portion 1062, wherein the first distal conduit end 1181 is connected directly or indirectly to an inert gas source, wherein the second distal conduit end 1182 is connected directly or indirectly to a vacuum pump, wherein the reactor 100 further comprises a three way valve 120 configured to monitor flow of both liquids and gasses.

According to some embodiments, the second distal conduit end is indirectly connected to a vacuum pump through a vacuum trap 122. Vacuum traps are conventional chemical lab glassware mediating between reaction vessels or chemical containers and vacuum pump. An example is shown in FIG. 1B.

With reference to the method of the present invention, according to some embodiments, inserting inert gas into the enclosure 103 through the conduit 116 entails switching the valve 120 from a gas blocking state, in which the first distal conduit 1181 end is in fluid isolation from the bottom internal cavity portion 1062 to a gas transferring state, in which the first distal conduit end 1181 is in fluid communication with the bottom internal cavity portion 1062. According to some embodiments, applying vacuum by the vacuum pump to the bottom internal cavity portion 1062 entails operating the vacuum pump and switching the valve 120 from a liquid blocking state, in which the second distal 1182 conduit end is in fluid isolation from the bottom internal cavity portion 1062 to a liquid transferring state, in which the second distal conduit end 1182 is in fluid communication with the bottom internal cavity portion 1062.

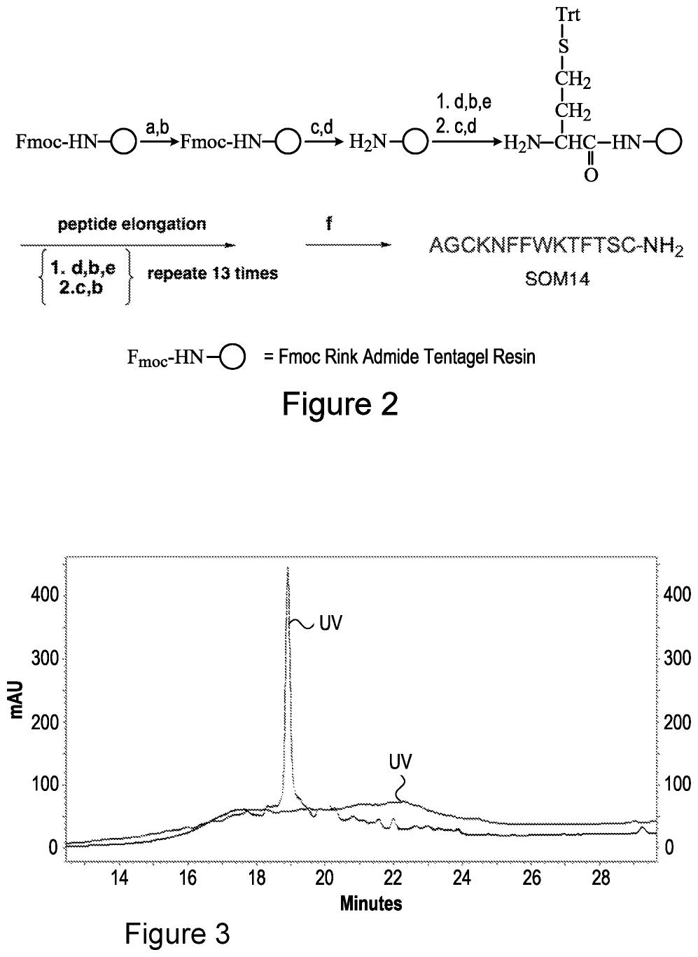

According to some embodiments, the reactor 100 further comprises a second conduit 124, for introducing chemicals, such as the starting materials of the present method into the internal cavity 106. As shown in FIG. 2B, second conduit 124 may be inserted into the internal cavity 106 through a second opening 109 located at the top enclosure face 1033. FIG. 2B further shows a syringe pump 126, configured to introduce materials into the internal cavity 106 through second conduit 124, at a predetermined controlled rate. According to some embodiments, step iii. of inserting at least one reactant into the reaction chamber entails providing the reactant through the second conduit 124. According to some embodiments, the reactant is provided as a flowable composition, and step iii. entails flowing the reactant through the second conduit 124. According to some embodiments, the reactant is provided as a liquid composition, and step iii. entails flowing the liquid composition through the second conduit 124 into the reaction chamber. According to some embodiments, the reactant is provided as a liquid solution, and step iii. entails flowing the liquid solution through the second conduit 124 into the reaction chamber.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

### EXAMPLES

### Example 1-Extremely Fast Solid Phase Synthesis of Somatostatin 14 (SEQ ID NO. 5)

### General Procedure

All peptides were synthesized on solid support from carboxy to amino terminus, utilizing Fmoc chemistry for the N^{α} protection. Fmoc (Fluorenylmethyloxycarbonyl) Rink Amide methylbenzhydrylamine (MBHA) resin with loading capacity of 0.71 mmol/g was used for all the reactions in this work. The resin quantity was 10% w/v (5 gr resin/50 mL solvent). All the reactions were carried out in a jacketed 100 mL glass reactor (FIG. 1) at 50° C. under nitrogen. All stirrings were performed by an overhead stirrer at 1200 ppm. The equivalents of all reagents used were calculated in respect to the resin loading capacity using 2 MER (Molar Equivalent Ratio). Reaction times of each one of steps (c) and (d) was 30 seconds. A general scheme of the process sequence is depicted in FIG. 2. Steps (a)-(f) in FIG. 2 and in the present example indicate as follows:
(a) Swelling; (b) Washing; (c) Fmoc deprotection; (d) Coupling; (e) Sampling for small cleavage; and (f) Final cleavage and deprotection. The steps are further detailed below.

### Detailed Procedures

All couplings and washings were performed in pre-heated (50° C.) NMP (N-Methyl-2-pyrrolidone)

All couplings were performed under basic conditions utilizing DIPEA (N,N-Diisopropylethylamine, 8 eq/coupling).
(a) Swelling—was conducted 3 times in each occurrence for 2 minutes in NMP at 50° C. and at stirring rate of 1200 rpm.
(b) Washing—was conducted 3 times in each occurrence for 0.5 minutes using NMP at 50° C. and at stirring rate of 1200 rpm.
(c) Fmoc deprotection—was conducted using a solution of 5% piperidine in NMP pre heated to 50° C. for 0.5 minutes at stirring rate of 1200 rpm.
(d) Coupling—40 mL of NMP pre heated to 50° C., was added to the reactor and stirred. After the addition of the protected amino acid and 8 eq of DIPEA to the reactor, a solution of HATU (1-[Bis(dimethylamino)methylene]-1H-1,2,3-triazolo[4,5-b]pyridinium 3-oxide hexafluorophosphate) in NMP was added over ½ min. Following the addition, the reaction mixture was stirred for ½ min at 1200 rpm.
(c) Small cleavage—a small sample of resin was collected using a small spatula after the coupling, washings, and draining, and put in an Eppendorph. Fmoc was deprotected by the addition of few drops of 20% piperidine. After 1 min vortex the resin was washed by decantation with isoprponal (3 times, 2 minutes each) and ether (twice, 2 minutes each). The resin was dried by steam of N₂ and a 1 ml pre-cooled solution (to 0° C.) of TFA (trifluoroacetic acid, 95%), distilled water (TDW) (2.5%), and TIS (triisopropylsilane 2.5%) was added. The mixture was vortexed for 30 minutes at room temperature. The TFA was fully evaporated by a stream of nitrogen. The remained crude peptide was dissolved in 2 mL ACN/TDW (1:1), filtered, and analyzed using analytical HPLC/MS. It is to be understood that this stage is performed for the monitoring of the reaction.
(f) Final cleavage/Deprotection—The NMP-wet protected peptidyl-resin in the reactor, was first washed, with stirring, three times by 30 mL of DCM (dichloromethane), drained and was subsequently charged with 40 mL of cold cleavage/global deprotection solution at 10° C. consisting of 92.5% TFA, 2.5% TIS, and 5% H₂O. The reaction temperature was increased to ambient temperature after the addition and the reaction mixture was stirred for 2 h. under low stream of N₂. The cleaved resin was then filtered off and rinsed with a minimal amount of TFA. A total of 70 mL of cold diethyl ether (−20° C.) was added slowly to the combined rinsing peptide/TFA solution followed by the addition of another 70 mL of cold diethyl ether (−20° C.) within 5 min. The mixture was stirred for 15 min. The crude peptide product precipitated in this process. The crude peptide was collected by filtration, and the filter cake was rinsed four times with 7 mL of cold (−20° C.) diethyl ether and was dried at 30° C. under vacuum overnight.

### Analytical HPLC

HPLC analyses were performed on a Waters e2695 system equipped with a pump, 2489 UV/Vis variable wavelength detector recording, and a column. Chromatograms were recorded at 280 nm at room temperature with a flow rate of 1 mL/min. The mobile phase consisted of solution A: TDW (0.1% v/v TFA) and solution B: ACN (acetonitrile, 0.1% v/v TFA). The detailed HPLC gradient program is presented below. The collected fractions were analyzed by MS. To obtain analytical HPLC chromatograms of crude peptides, all samples were dissolved in TDW/ACN 1:1 mixture, filtered through a 0.45 μm PTFE filters and injected to a reversed phase analytical HPLC column of Waters (XSELECTTM CSHTM 130 Å C18, 4.6 mm×150 mm, 3.5 μm). The detailed HPLC eluent composition program versus time is depicted in Table 1.

TABLE 1
Analytical HPLC program

| Time (min) | % TDW (0.1% TFA) | % ACN (0.1% TFA) |
|---|---|---|
| 0 | 95 | 5 |
| 1 | 95 | 5 |
| 6 | 78 | 22 |
| 16 | 68 | 32 |
| 20 | 5 | 95 |
| 22 | 5 | 95 |
| 24 | 95 | 5 |
| 26 | 95 | 5 |

### Results

### HPLC

The HPLC results chromatogram of the reaction product produced as detailed above is presented in FIG. 3. The molecular weight of the peak was measured to be 1640 by MS.

### HPLC Mass Spectrometry Analyses

Mass spectra were gained on LCQ Fleet Ion Trap mass spectrometer (Thermo Scientific) utilizing electrospray ionization. For high resolution mass spectrometry (HRMS) analyses, the spectra were recorded on Agilent 6550 iFunnel Q-TOF LC/MS system.

MW of SOM14—Found: 1640 calc (M+2): 1640.

### Microscope Bead Characterization

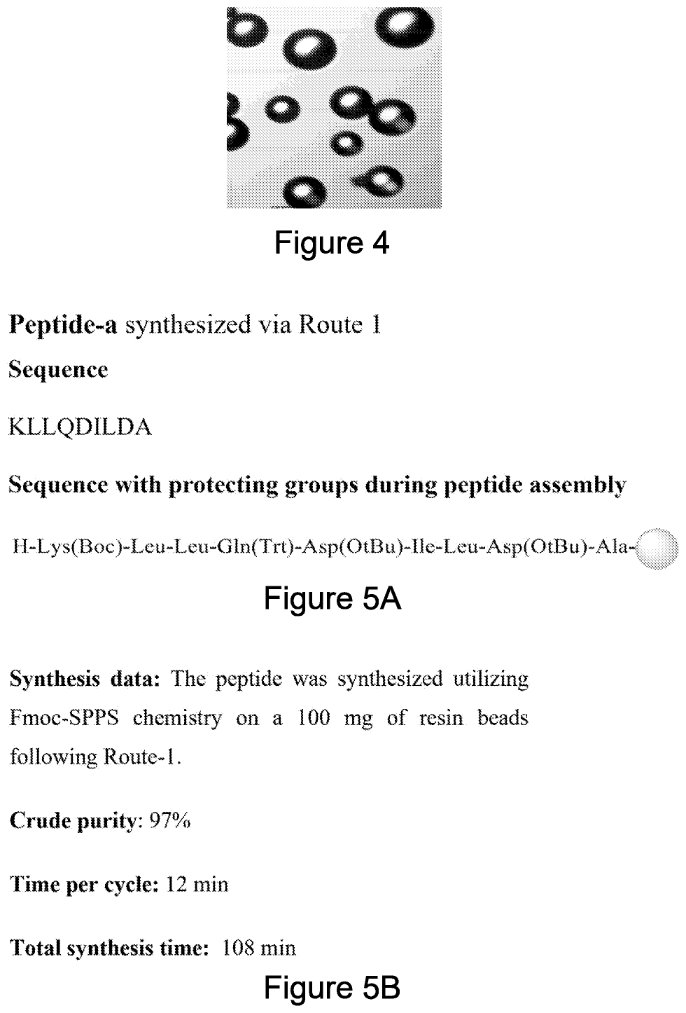

A small portion of the resin beads after the synthesis of SOM14 (somatostatin 14, SEQ ID NO. 5), washings, and drying was put on glass slide under the microscope (ZEISS Scope.A1 with AxioCamm ICc 3). The resin beads measured with Objectives zoom of 5 and 20. The measured beads are spherical and intact with approximate diameter of 100 μm. FIG. 4 is a close-up photograph of the resin beads at the end of the process presented above.

### Examples 2-6: High-Temperature Fast Stirred Peptide Synthesis (HTFSPS)

### Materials and Methods

### Materials

9-fluorenylmethyloxycarbonyl-N^{α}-protected amino acids (Fmoc-N^{α}-XX-OH): Fmoc-Ala-OH·H2O, Fmoc-Arg(Pbf)-OH; Fmoc-Asn(Trt)-OH; Fmoc-Asp-(OtBu)-OH; Fmoc-Cys(Trt)-OH; Fmoc-(D)-Cys(Trt)-OH Fmoc-Gln(Trt)-OH; Fmoc-Gly-OH; Fmoc-His(Trt)-OH; Fmoc-(D)-His(Trt)-OH; Fmoc-Ile-OH; Fmoc-Leu-OH; Fmoc-Lys(Boc)-OH; Fmoc-Met-OH; Fmoc-Orn(Boc)-OH; Fmoc-Phe-OH; Fmoc-Pro-OH; Fmoc-Ser(tBu)-OH; Fmoc-Thr(tBu)-OH; Fmoc-Trp(Boc)-OH; Fmoc-Tyr(tBu)-OH; 1-[Bis(dimethylamino)methylene]-1H-1,2,3-triazolo[4,5-b]pyridinium3-oxid hexafluorophosphate (HATU), were purchased from Chem-Impex International Inc. (Wood Dale, IL, U.S.A.). Fmoc-Rink-Amide methylbenzhydrylamine (MBHA) resin (200-400 mesh, 0.71 mmol/g resin) was purchased from Iris Biotech GmbH (Marktredwitz, Germany). Diethyl ether, piperidine, trifluoroacetic acid (TFA), N,N-diisopropylethylamine (DIPEA), methanol (MeOH), triisopropylsilane (TIS), were purchased from ACROS ORGANICS N.V. (Geel, Belgium). Organic solvents for solid phase synthesis (SPS) and for high performance liquid chromatography (HPLC) including: N,N-dimethylformamide (DMF), dichloromethane (DCM), and acetonitrile (ACN) were purchased from Bio-Lab (Jerusalem, Israel). Water for HPLC and other laboratory analyses was distilled (three distilled water, TDW) by MilliQ water system (Millipore, Merck).

### Synthetic Methods

HTFSPS reactor set-up: HTFSPS reactions were performed in a 20 mL glass vessel with sintered glass bottom, switch control for waste pumping, heating jacket, and overhead mixing utilizing a straight five-bladed Teflon turbine and an engine (mrc RK 2200 Digital Overhead Stirrer) capable of mixing in the range of 50-2200 rpm (FIGS. 1A-C). The impeller was placed 3 mm above the swelled resin bed. A Teflon feed pipeline equipped with a female luer was inserted through the reactor cap. All Reagent mixtures and washing solvents were added from the feed pipeline and drained by opening the Teflon valve connected to a vacuum pump. The feed pipeline was placed just above the turbine close to the inner reactor wall to reduce vortex formation and improve turbulent mixing.

### Solid Phase Peptide Synthesis Protocols

Solid support properties: Fmoc Rink Amide methylbenzhydrylamine (Fmoc-MBHA) resin with loading capacity of 0.71 mmol/g was used as the solid support for all the reactions in this study yielding C-terminal amide after cleavage. Peptides were synthesized from carboxy to amino terminus utilizing the Fmoc group for N^{α} protection. The equivalents of all reagents were calculated according to loading capacity of 0.071 mmol for 100 mg resin. Resin beads were pre-swollen in DMF for 1 hour prior to initializing the synthesis.

### Synthetic Routes Used for the Synthesis of Each Peptidyl Sequences

Route-1, Route-2, and Route-3 were applied for synthesis of peptide-a (Example 2, SEQ ID NO. 1).
Route-2, Route-3, Route-4, Route-5, Route-6, and Route-MW were applied for synthesis of SOM (Example 5, SEQ ID NO. 5).
Route-3 was applied for synthesis of: HFGWI (Example 3, SEQ ID NO. 2); and HFG; DHFG; VAS (SEQ ID NO. 3); and DDVAS (SEQ ID NO. 4) (Example 4).
Route-6 was applied for synthesis of MARADONA (SEQ ID NO. 6, Example 6).

### Cycle Conditions

In HTFSPS, coupling reagents solutions of amino acid, HATU and DIPEA were mixed and added to the reactor immediately without pre-heating or pre-activation. In MW-SPPS, coupling reagents solutions of amino acid, HATU and DIPEA were added sequentially to the reactor without pre-heating or pre-activation.

TABLE 2
Route-1 HTESPS

| Reaction/step | Conditions |
|---|---|
| Fmoc removal | The resin was incubated for 5 min with 3 mL solution of 5% piperidine |
| | in DMF applying constant stirring of 1200 rpm at 30° C. |
| Wash | 2 × 3 mL DMF for 30 sec each |
| Coupling (amide | The resin was incubated for 5 min with 3 mL of DMF solution |
| bond formation) | containing: 2 equiv. AA, 2 equiv. HATU, and 8 equiv. DIPEA applying |
| | constant stirring of 1200 rpm at 30° C. |
| Wash | 2 × 3 mL DMF for 30 sec each |
| Wash at the | 2 × 3 mL DMF, 2 × 3 mL DCM, 2 × 3 mL diethylether for 30 sec each |
| overall synthesis | |

TABLE 3
Route-2 HTFSPS

| Reaction/step | Conditions |
|---|---|
| Fmoc removal | The resin was incubated for 30 sec with 3 mL solution of 5% piperidine |
| | in DMF applying constant stirring of 1200 rpm at 30° C. |
| Wash | 2 × 3 mL DMF for 30 sec each |
| Coupling (amide | The resin was incubated for 30 sec with 3 mL of DMF solution |
| bond formation) | containing: 2 equiv. AA, 2 equiv. HATU, and 8 equiv. DIPEA applying |
| | constant stirring of 1200 rpm at 30° C. |
| Wash | 2 × 3 mL DMF for 30 sec each |
| Wash at the | 2 × 3 mL DMF, 2 × 3 mL DCM, 2 × 3 mL diethylether for 30 sec each |
| overall synthesis | |

TABLE 4
Route-3 HTFSPS

| Reaction/step | Conditions |
|---|---|
| Fmoc removal | The resin was incubated for 30 sec with 3 mL solution of 5% piperidine |
| | in DMF applying constant stirring of 1200 rpm at 90° C. |
| Wash | 2 × 3 mL DMF for 30 sec each |
| Coupling (amide | The resin was incubated for 30 sec with 3 mL of DMF solution |
| bond formation) | containing: 2 equiv. AA, 2 equiv. HATU, and 8 equiv. DIPEA applying |
| | constant stirring of 1200 rpm at 90° C. |
| Wash | 2 × 3 mL DMF for 30 sec each |
| Wash at the | 2 × 3 mL DMF, 2 × 3 mL DCM, 2 × 3 mL diethylether for 30 sec |
| overall synthesis | each. Note: The HTFSPS reactor was cooled down to room |
| | temperature before final washes with DCM and ether |

TABLE 5
Route-4 HTFSPS

| Reaction/step | Conditions |
|---|---|
| Fmoc removal | The resin was incubated for 30 sec with 3 mL solution of 5% piperidine |
| | in DMF applying constant stirring of 100 rpm at 90° C. |
| Wash | 2 × 3 mL DMF for 30 sec each |
| Coupling (amide | The resin was incubated for 30 sec with 3 mL of DMF solution |
| bond formation) | containing: 2 equiv. AA, 2 equiv. HATU, and 8 equiv. DIPEA applying |
| | constant stirring of 100 rpm at 90° C. |
| Wash | 2 × 3 mL DMF for 30 sec each |
| Wash at the | 2 × 3 mL DMF, 2 × 3 mL DCM, 2 × 3 mL diethylether for 30 sec each. |
| overall synthesis | Note: The HTFSPS reactor was cooled down to room temperature |
| | before final washes with DCM and ether |

TABLE 6
Route-5 HTFSPS

| Reaction/step | Conditions |
|---|---|
| Fmoc removal | The resin was incubated for 20 sec with 3 mL solution of 20% piperidine |
| | in DMF applying constant stirring of 1200 rpm at 90° C. |
| Wash | 1 × 3 mL DMF for 10 sec each |
| Coupling (amide | The resin was incubated for 20 sec with 3 mL of DMF solution |
| bond formation) | containing: 6 equiv. AA, 6 equiv. HATU, and 10 equiv. DIPEA applying |
| | constant stirring and of 1200 rpm at 90° C. |
| Wash | 1 × 3 mL DMF for 10 sec each |
| Wash at the | 2 × 3 mL DMF, 2 × 3 mL DCM, 2 × 3 mL diethylether for 30 sec |
| overall synthesis | each. Note: The HTFSPS reactor was cooled down to room |
| | temperature before final washes with DCM and ether |

TABLE 7
Route-6 HTFSPS

| Reaction/step | Conditions |
|---|---|
| Fmoc removal | The resin was incubated for 10 sec with 3 mL solution of 20% piperidine |
| | in DMF applying constant stirring of 1200 rpm at 90° C. |
| Wash | 1 × 3 mL DMF for 10 sec each |
| Coupling (amide | The resin was incubated for 10 sec with 3 mL of DMF solution |
| bond formation) | containing: 6 equiv. AA, 6 equiv. HATU, and 10 equiv. DIPEA applying |
| | constant stirring and of 1200 rpm at 90° C. |
| Wash | 1 × 3 mL DMF for 10 sec each |
| Wash at the | 2 × 3 mL DMF, 2 × 3 mL DCM, 2 × 3 mL diethylether for 30 sec each |
| overall synthesis | Note: The HTFSPS reactor was cooled down to room temperature |
| | before final washes with DCM and ether |

TABLE 8
Route-2 MW-SPPS on a Liberty Blue microwave-
assisted peptide synthesizer (CEM))

| Reaction/step | Conditions |
|---|---|
| Fmoc removal | The resin was incubated with 5 mL solution of 20% piperidine in DMF |
| | at for 25 sec at 75° C. followed by additional 50 sec at 90° C. |
| Wash | 3 × 5 mL DMF for 20 sec each |
| Coupling (amide | The resin was incubated with 5 mL of DMF solution containing: 5 |
| bond formation) | equiv. AA, 5 equiv. HATU, and 10 equiv. DIPEA for 25 sec at 75° C. |
| | followed by additional 110 sec at 90° C. Note: in AA cysteine |
| | couplings the incubation lasted 120 sec at RT, elevated to 50° C. within |
| | 10 sec, and remained constant for 470 sec. |
| Wash | 3 × 5 mL DMF for 20 sec each |
| Wash at the | 2 × 5 mL DMF, 2 × 5 mL DCM, 2 × 5 mL diethylether for 30 sec |
| overall synthesis | each |

Cleavage from the solid support: For cleaving of the crude synthesized peptide from solid support and removal of all side chains protecting groups, few dried beads of the final peptidyl-resin were dissolved in a 2 ml pre-cooled solution (at 0° C.) composed of TFA (95%), three distilled water (TDW) (2.5%), and triisopropylsilane (TIS) (2.5%). The mixture was kept standing for 30 minutes in an ice bath, and then was shaken for another 150 minutes at room temperature. The TFA filtrate (including the cleaved peptide) was partially evaporated by a stream of nitrogen, and cold diethyl ether was added to the remained solution to remove the scavengers and other hydrophobic impurities, while the crude peptide was precipitated by centrifugation. Diethyl ether was then removed by decanting. The precipitation and decanting were repeated twice. Next, the residual ether was allowed to evaporate and the dry crude peptide was dissolved in ACN/TDW (1:1). The solution was frozen by liquid nitrogen and lyophilized overnight to be later analyzed by analytical HPLC/MS.

### Analysis and Characterization

Analytical high performance liquid chromatography (HPLC): Analytical HPLC analyses were performed on a Merck-Hitachi system equipped with an L-7100 pump, L-7400 UV detector, and a column. all samples were dissolved in TDW/ACN 50:50 mixture excluding VAS, DDVAS (Example 4), and MARADONA (Example 6), which were dissolved in TDW/ACN 95:5, filtered through a 0.22 μm PTFE filters, and injected to a reversed phase analytical HPLC column. Chromatograms were recorded at 220 nm at room temperature with a flow rate of 1 mL/min. The mobile phase consisted of solution A: TDW (0.1% v/v TFA) and solution B: ACN (0.1% v/v TFA). The HPLC conditions are presented below. The collected fractions were analyzed by ESI-MS. Crude purity of each peptide was calculated by integration of the desired peak detected by ESI-MSt.

Analytical HPLC conditions used for peptide-a, SOM (Example 5), HFGWI (Example 3); HFG and DHFG (Example 4): The analytical HPLC with gradient of 5:95 to 45:55 ACN:TDW in 40 min utilizing Phenomenex Gemini RP C18 column (4.6 mm×150 mm) with a particle size of 5 μm 110A. (S/NO 289054-6, P/NO 00F-4435-E0).

Analytical HPLC conditions used for VAS (Example 4): The analytical HPLC with gradient of 5:95 to 40:60 ACN:TDW in 30 min utilizing Phenomenex Gemini RP C18 column (4.6 mm×150 mm) with a particle size of 5 μm 110A. (S/NO 289054-6, P/NO 00F-4435-E0).

Analytical HPLC conditions used for MARADONA (Example 6): The analytical HPLC with gradient of 2:98 to 30:70 ACN:TDW in 20 min utilizing Purospher STAR RP C18 endcapped column (4.6 mm×250 mm) with a particle size of 5 μm. (S/NO 946096, Sorbent Lot No. HX90393769).

Mass spectrometry analyses: Mass spectra were gained on LCQ Fleet Ion Trap mass spectrometer (Thermo Scientific) utilizing positive electrospray ionization (ESI-MS) in the mass range of 400-2000 m/z.

Microscopy: The microscope pictures were taken with zoom of ×100 in Axioskop 2 plus with DinoEye Eyepiece Camera. Resin beads before and after 7 hours challenge of stirring at 1200 rpm in the HTFS-SPPS reactor in addition to microscope pictures for all peptides after completing synthesis were placed on a glass slide and their size and integrity were characterized.

### Example 2—High-Temperature Fast Stirred Peptide Synthesis (HTFSPS) of a Nine-Amino Acid Model Peptide-a

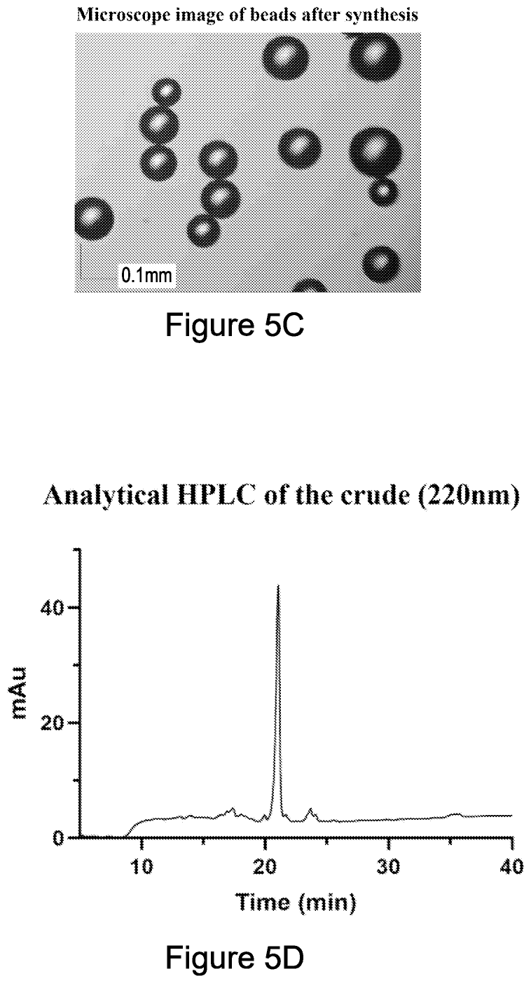

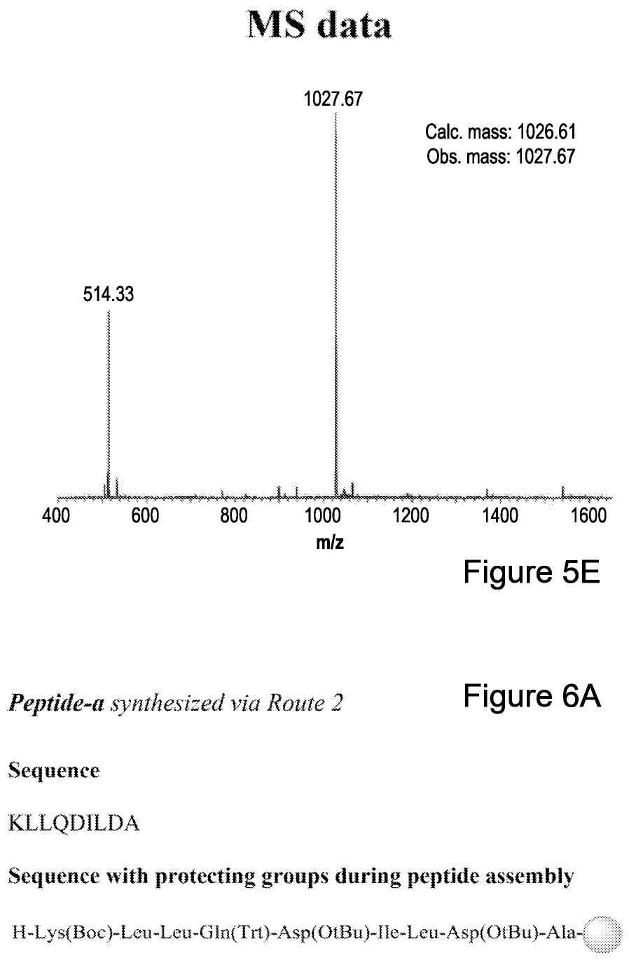

Initial assessment of HTFSPS feasibility was performed by synthesizing a nine-amino acid model peptide-a, KLLQDILDA (SEQ ID NO. 1, FIG. 1) at a constant stirring rate of 1200 rpm using the HTFSPS reactor, as shown in the figures. Peptide-a was synthesized in the HTFSPS reactor via several routes, which differ in the reaction conditions and the crude purity was determined after each synthesis. (Routes 1-3 above; FIGS. 5A-E, 6A-E and 7A-E). Reaction mixtures and washing solvents were added to the reactor by injection from the feed line and drained by vacuum filtration. Coupling mixtures containing only two equivalents of protected amino acid, an activator, and a base were added to the reactor without pre-heating or pre-activation. Fmoc deprotection was performed by inserting a solution of 5% piperidine in DMF without pre-heating. The crude purity of peptide-a synthesized via Route-1 (5 min reactions, 30° C.) was above 97% (FIGS. 5A-B).

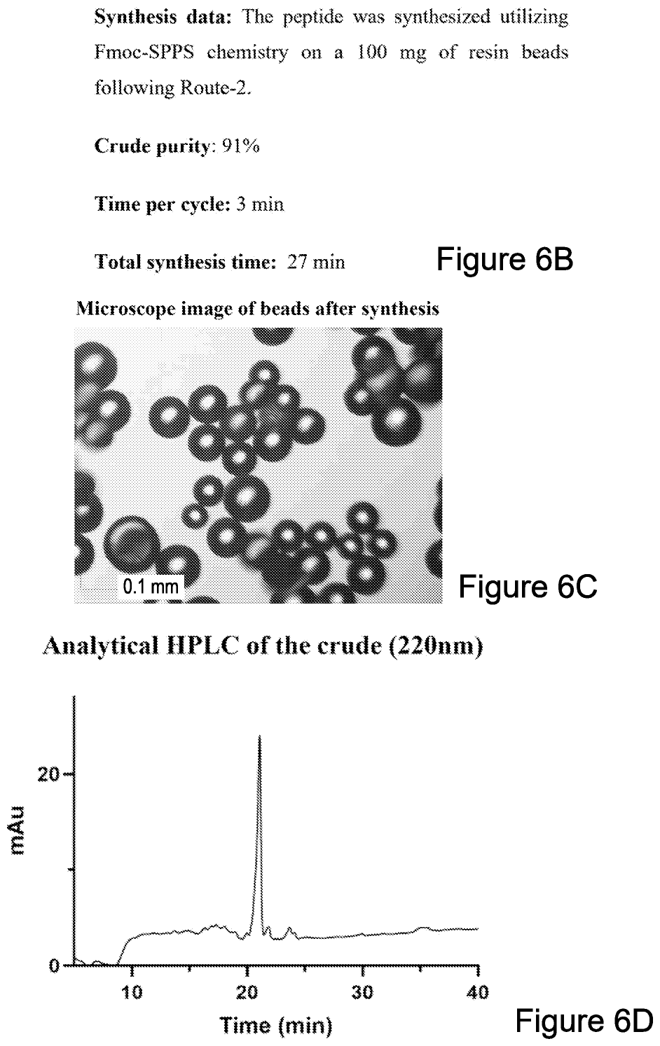

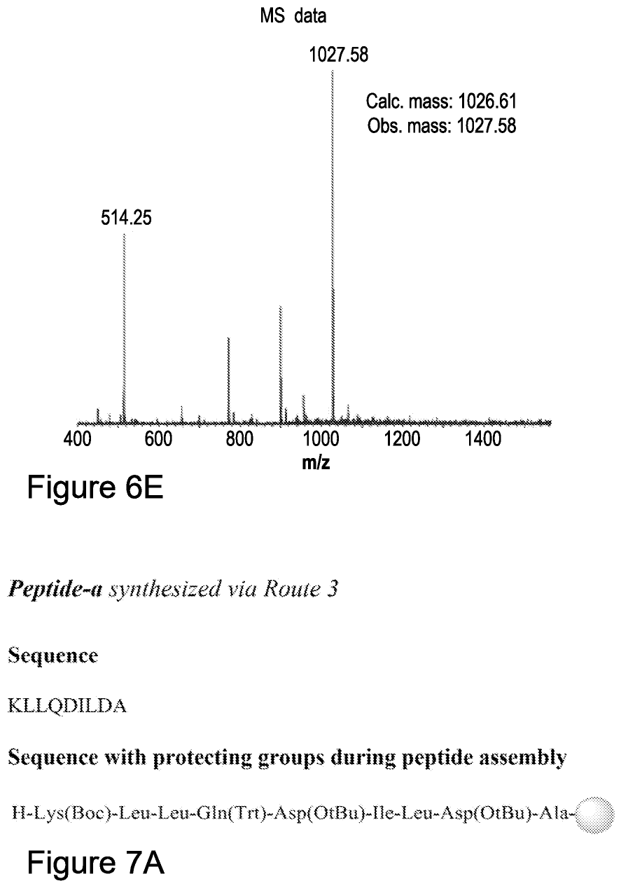

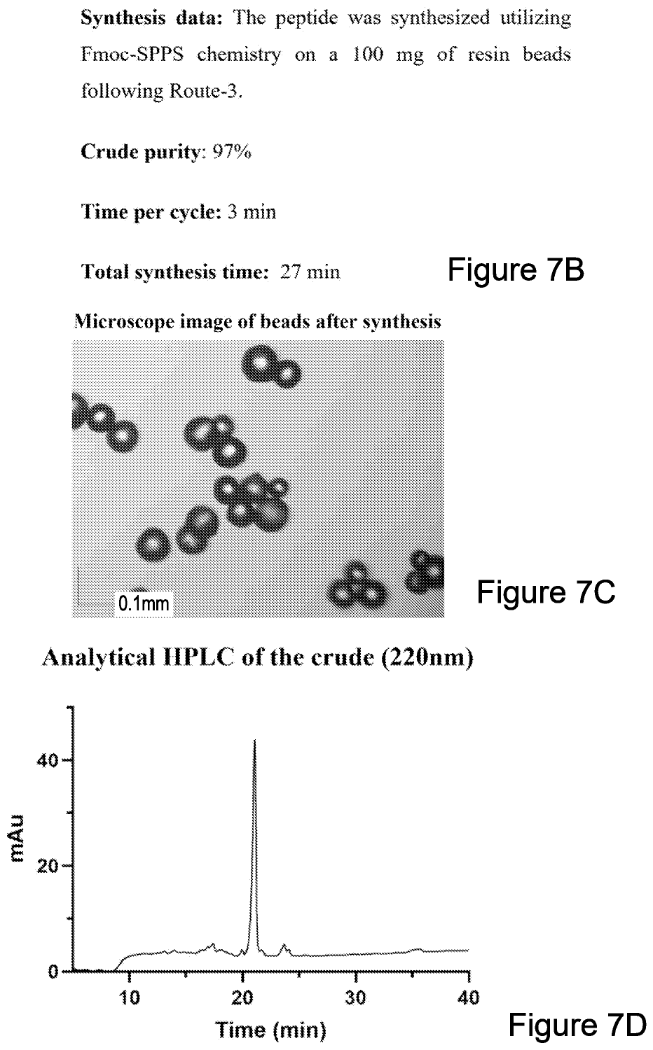

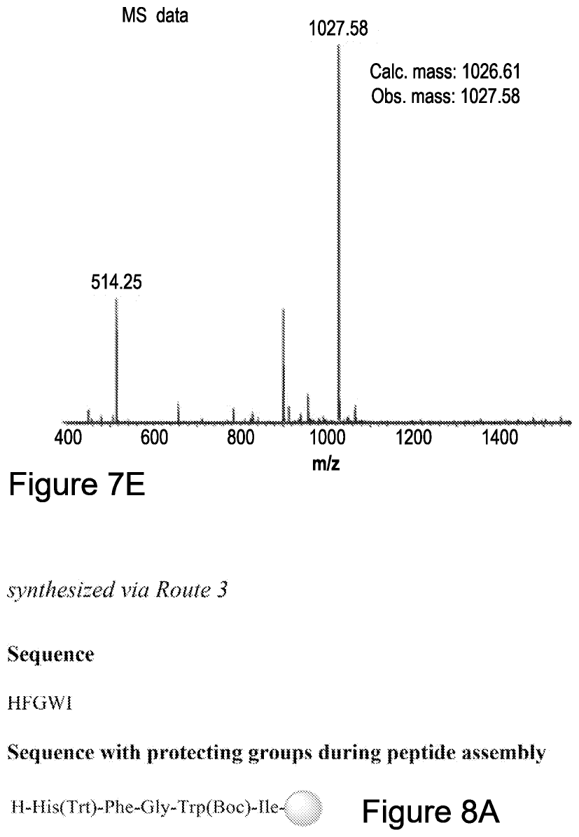

Furthermore, the result proves that given enough time, an almost complete conversion is achieved in each step when using HSS-SPPS even while using low concentrations of reagents. The crude purity of peptide-a synthesized via Route-2 (30 sec reactions, 30° C.) was 91% (FIGS. 6A-B). Since Route 2 applies much shorter reaction times compared to Route-1. it demonstrated that time is a limiting factor in reaching high conversion in each reaction step. Elevating the temperature to 90° C., while maintaining short reaction periods, fast stirring, and low reagent concentrations was conducted using Route-3 (FIGS. 7A-E). Peptide-a was synthesized via Route-3 (30 sec reactions, 90° C.), which is almost identical to Route-2, only that the entire process was performed at 90° C., instead of 30° C. (FIGS. 7A-B). The crude purity of peptide-a synthesized via Route-3 was above 97% and took only 27 min instead of 108 min via Route-1 with a similar outcome.

Surprisingly, this suggests that the combination of fast stirring and elevated temperature, enables by the HTFSPS reactor, can accelerate the SPPS process at low reagent concentrations. Advantageously, although the sequence contains two aspartic acids and the process was performed at a high temperature, no significant aspartimide formation was observed. Without wishing to be bound by any theory of mechanism of action, it is assumed that the combination of low piperidine concentration and a short reaction cycle decreases the probability of this side reaction.

### Example 3—High-Temperature Fast Stirred Peptide Synthesis (HTFSPS) of Difficult Coupling of Model Peptide HFGWI

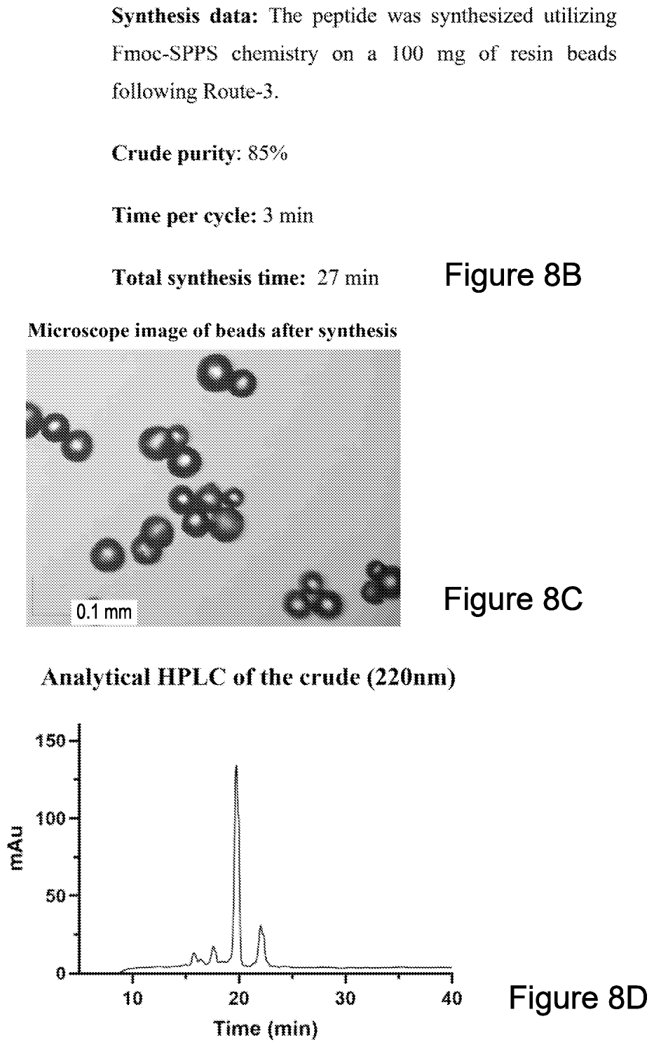

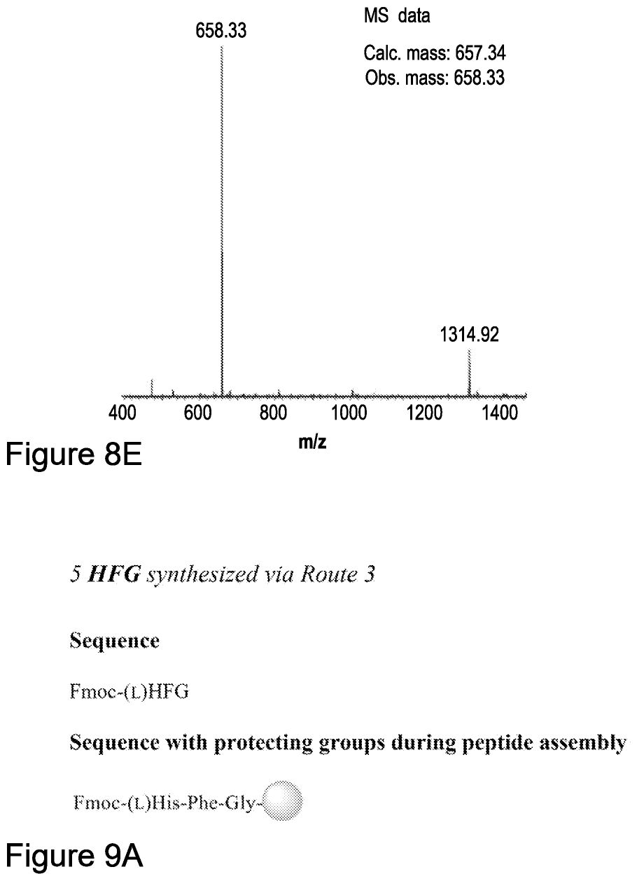

To check whether the HTFSPS can be used for difficult couplings, a notoriously hard coupling of Fmoc-L-His(Trt)-OH to the tetrapeptide H₂N-Phe-Gly-Trp-Ile was used here as a case study (FIGS. 8A-E, HFGWI, SEQ ID NO. 2) Coupling of Fmoc-L-His(Trt)-OH was performed in the HTFSPS-reactor using the same conditions described in Route-3, and the HPLC analysis confirmed over 85% conversion (FIG. 8B). The result is very encouraging, as a similar reaction performed for 60 minutes using 700 rpm stirring at room temperature provided 82% conversion (Naoum, J.; Alshanski, I.; Gitlin-Domagalska, A.; Bentolila, M.; Gilon, C.; Hurevich, M. Diffusion Enhanced Amide Bond Formation on Solid Support. Org. Process Res. Dev. 2019, 23, 2733-2739). This shows that a combination of elevated temperature and fast stirring can be used instead of extended reaction even for difficult coupling steps, indicating that HTFSPS might surpass other technologies. The joint contribution of elevated temperature and stirring rate in HTFSPS can be used to perform rapid reactions not only for hurdles-free peptide sequences like peptide-a (SEQ ID NO. 1) but also for difficult-to-synthesize ones.

### Example 4—High-Temperature Fast Stirred Peptide Synthesis (HTFSPS) without Significant Epimerization

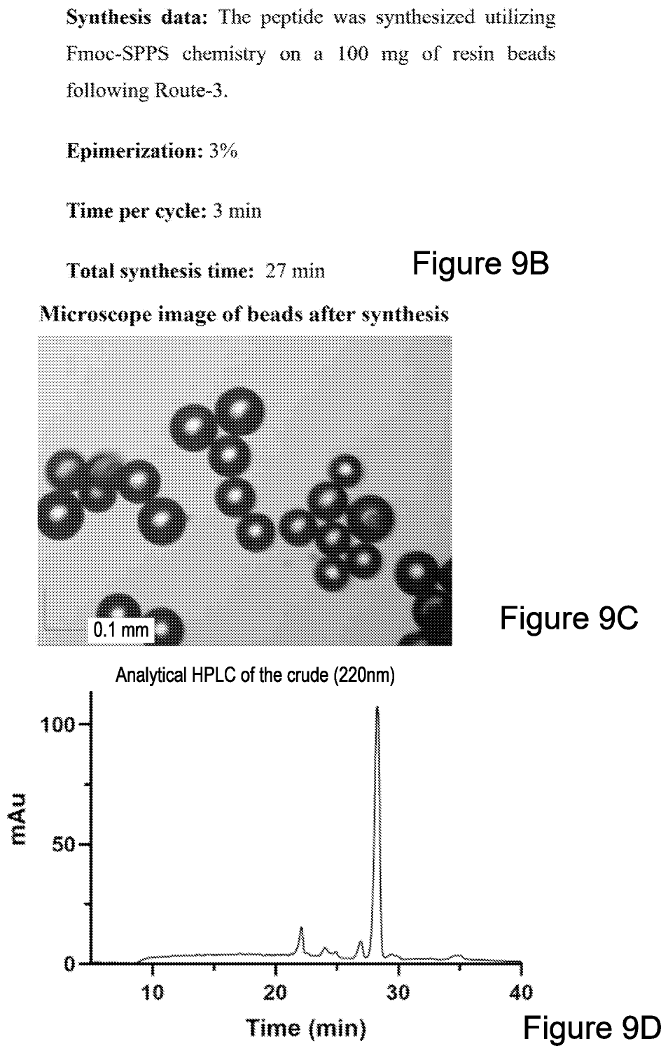

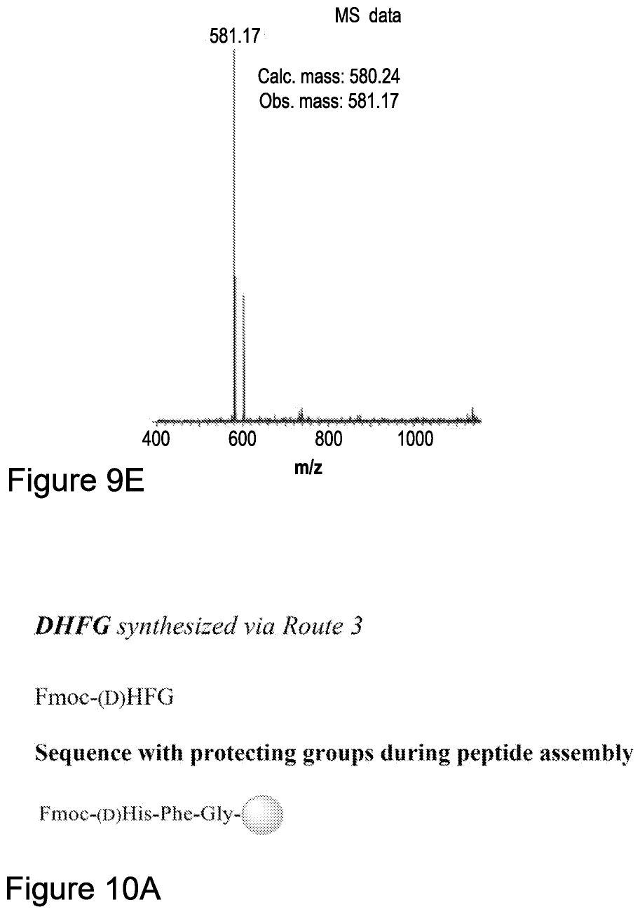

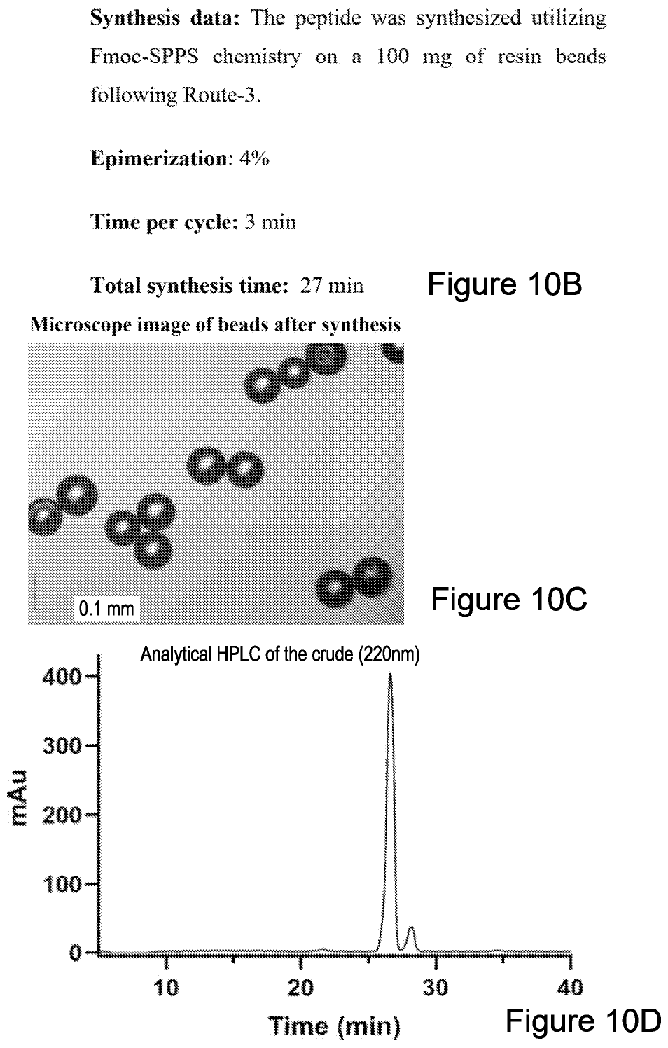

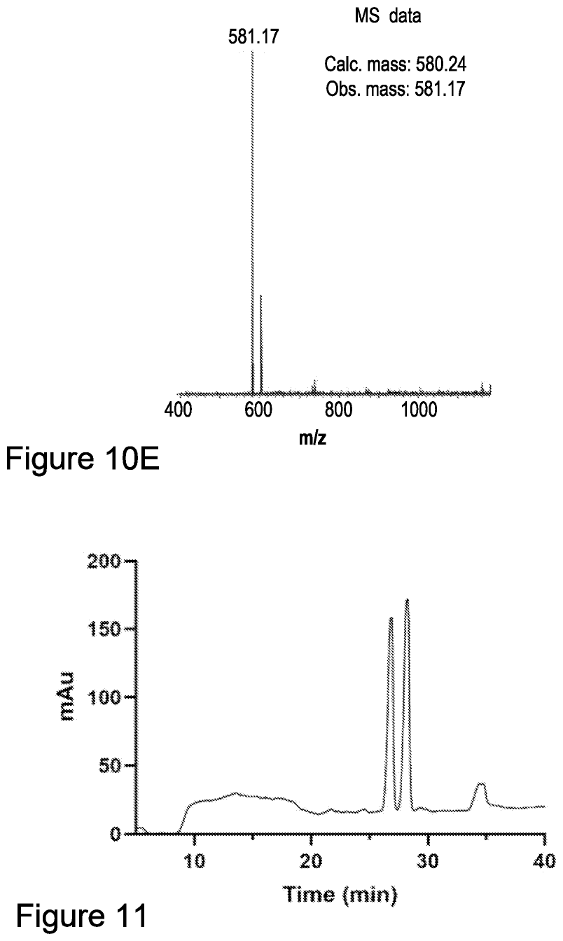

It is well accepted that elevated temperatures during coupling reactions can lead to racemization, especially of histidine, and also of cysteine. To evaluate if HTFSPS results in significant racemization, Fmoc-L-His(Trt)-OH was reacted with the Phe-Gly dipeptide under Route-3 conditions (FIGS. 9A-E, 10A-E and 11). The HPLC purity of tripeptide Fmoc-HFG and Fmoc-(D)HFG showed epimerization of less than 3% and 4%, respectively (FIGS. 9B and 10B). This specific coupling was chosen since it is prone to epimerization, and is often used as a model case study. Although the prone-to-epimerize Fmoc-L-His(Trt)-OH was used at a high temperature, the degree of epimerization was not significantly higher than in other methods.

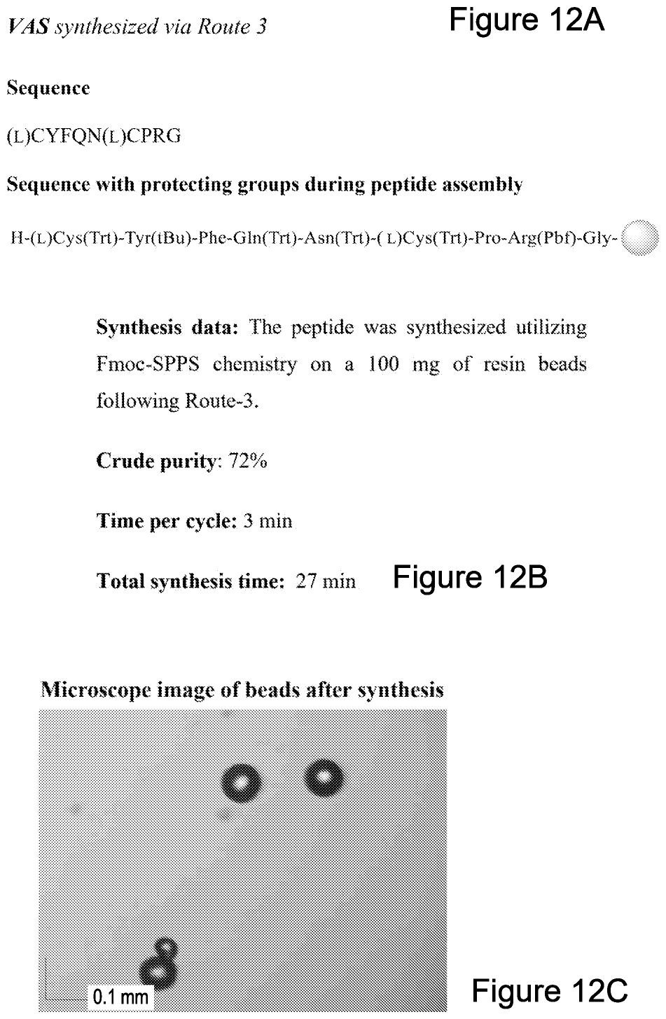

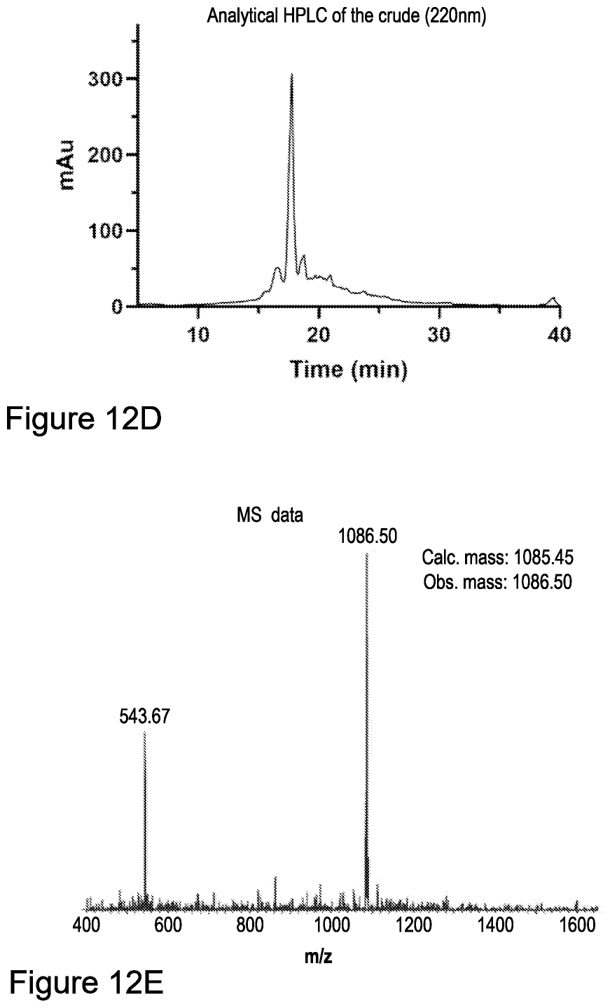

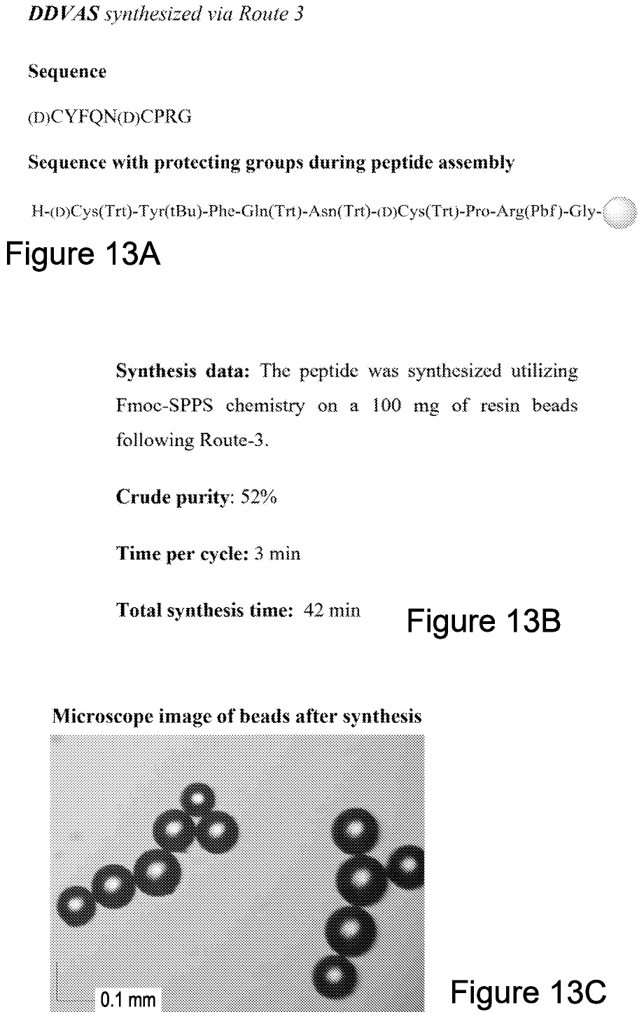

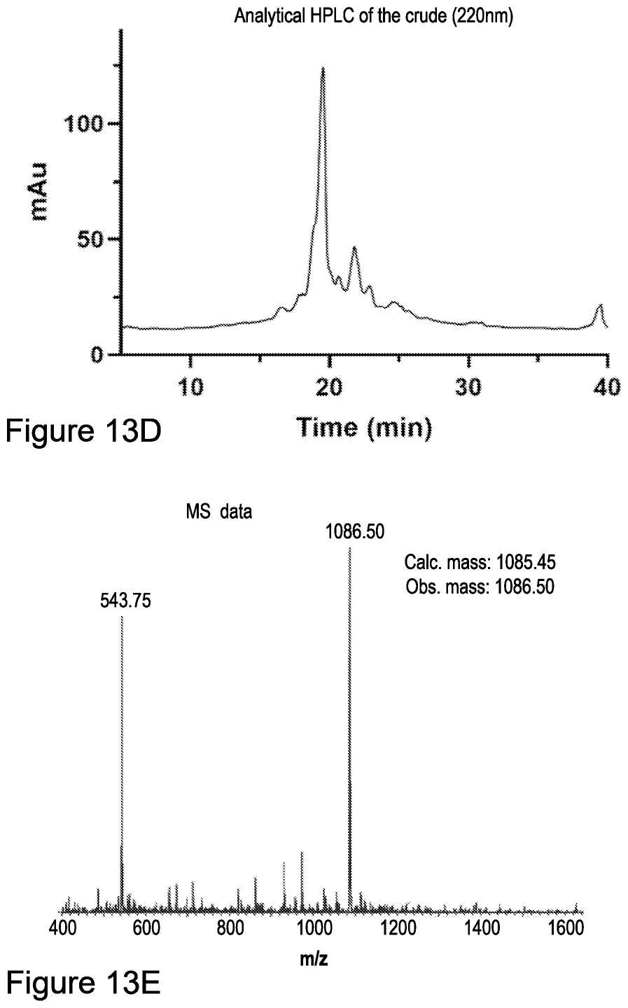

Two vasopressin-derived peptides VAS (with two L-Cys; SEQ ID NO. 3) and DDVAS (with two (D) Cys; SEQ ID NO. 4) were synthesized using HTFSPS via Route-3 to evaluate epimerization of cysteine. HPLC analysis showed that there were no significant traces of VAS in DDVAS and vice versa (FIGS. 12A-E and 13 A-E).

These studies indicate that HTFSPS does not result in significant epimerization compared to other methods (Mijalis, A. J.; Thomas, D. A.; Simon, M. D.; Adamo, A.; Beaumont, R.; Jensen, K. F.; Pentelute, B. L. A Fully Automated Flow-Based Approach for Accelerated Pep-tide Synthesis. Nat. Chem. Biol. 2017, 13 (5), 464-466). Without wishing to be bound by any theory of mechanism of action, it is assumed that the short time and the absence of pre-heating minimize racemization even at elevated temperatures. The above results confirm that, surprisingly, peptides containing His and Cys can be synthesized by HTFSPS without using special building blocks or deviating from the standard cycle protocol maintaining the high temperature.

### Example 5-High-Temperature Fast Stirred Peptide Synthesis (HTFSPS) of a Somatostatin Model

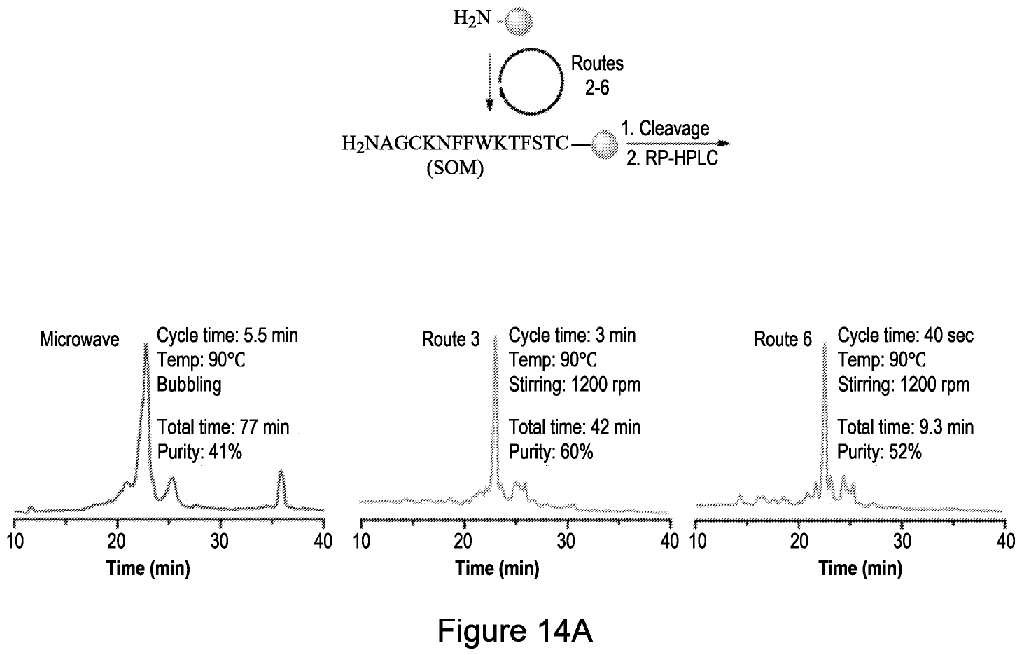

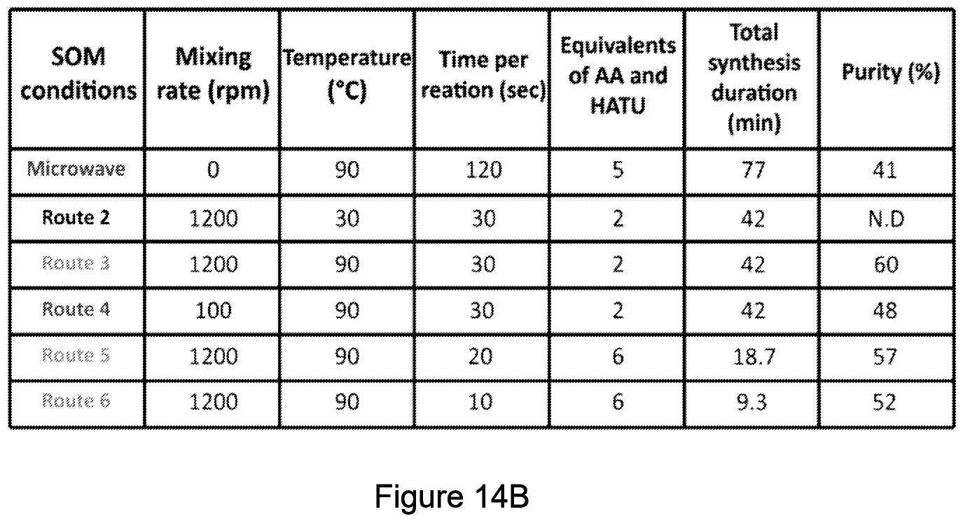

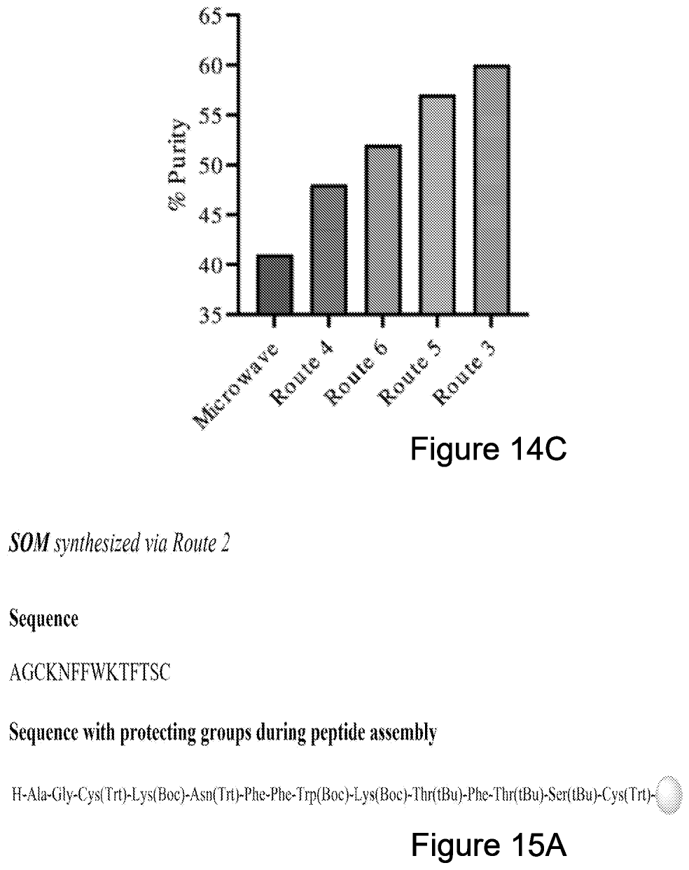

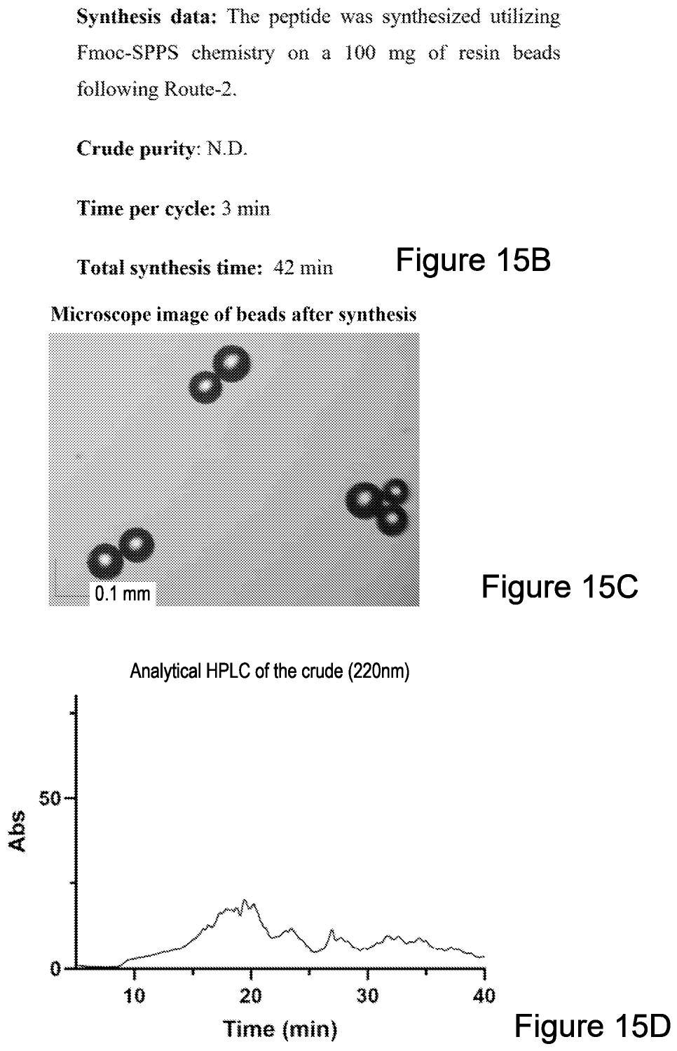

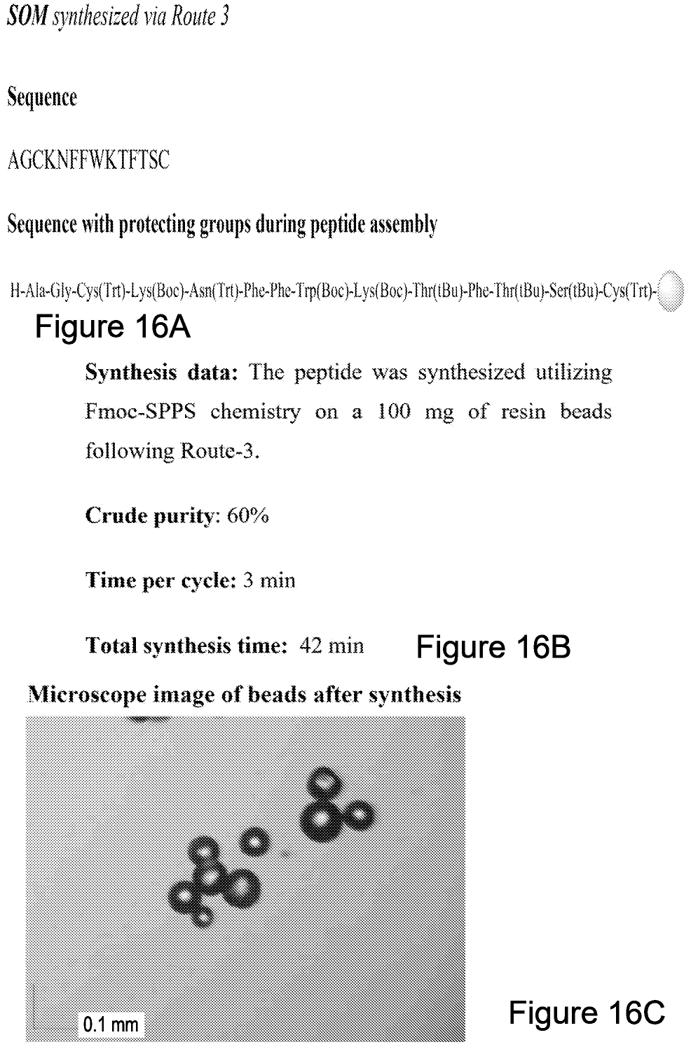

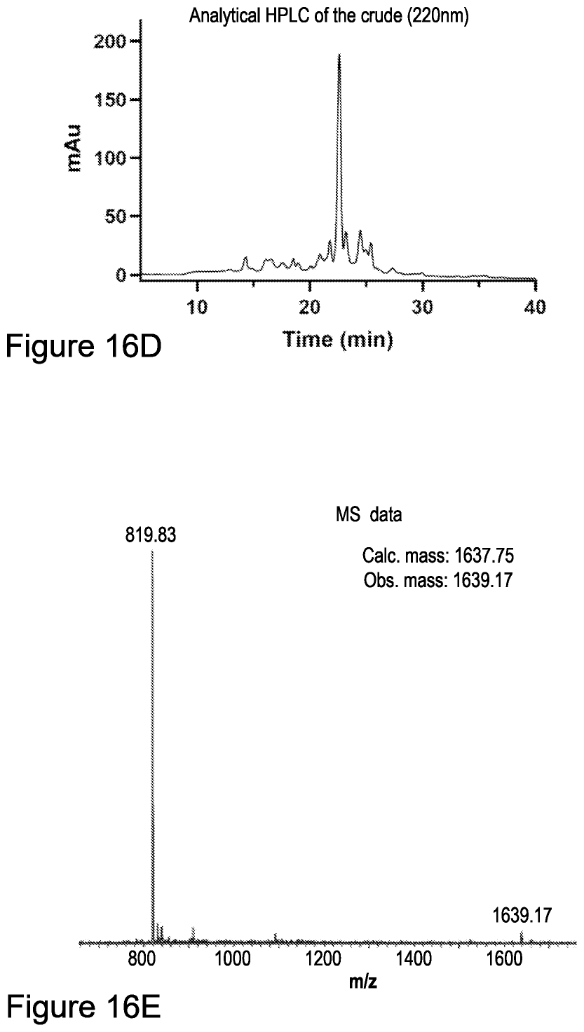

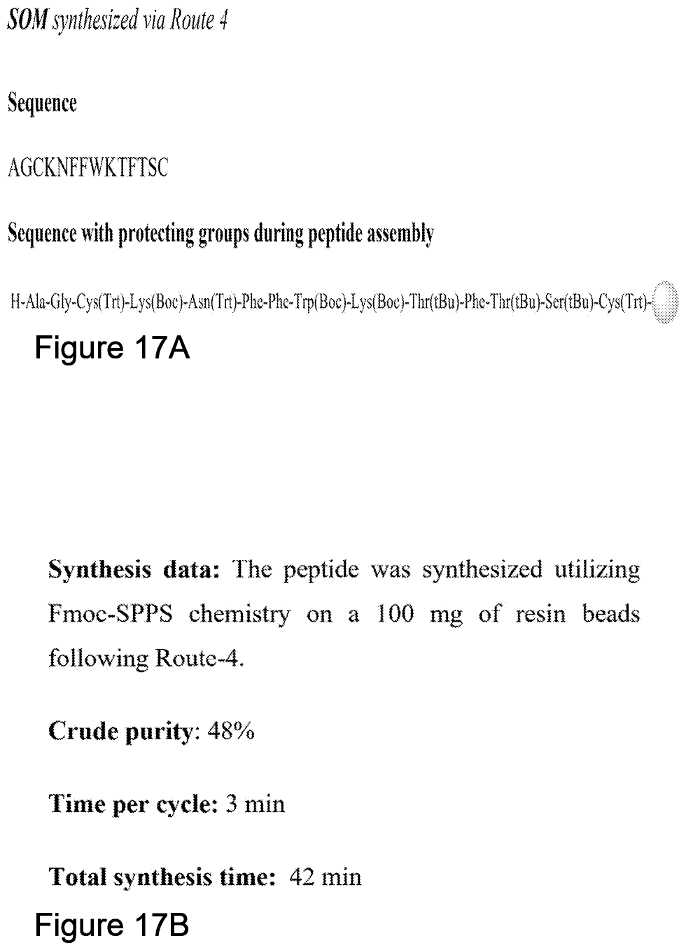

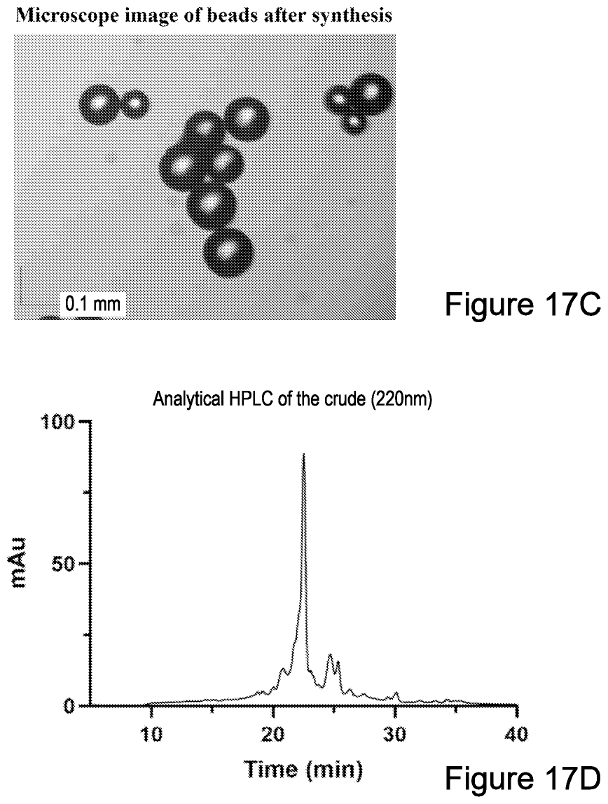

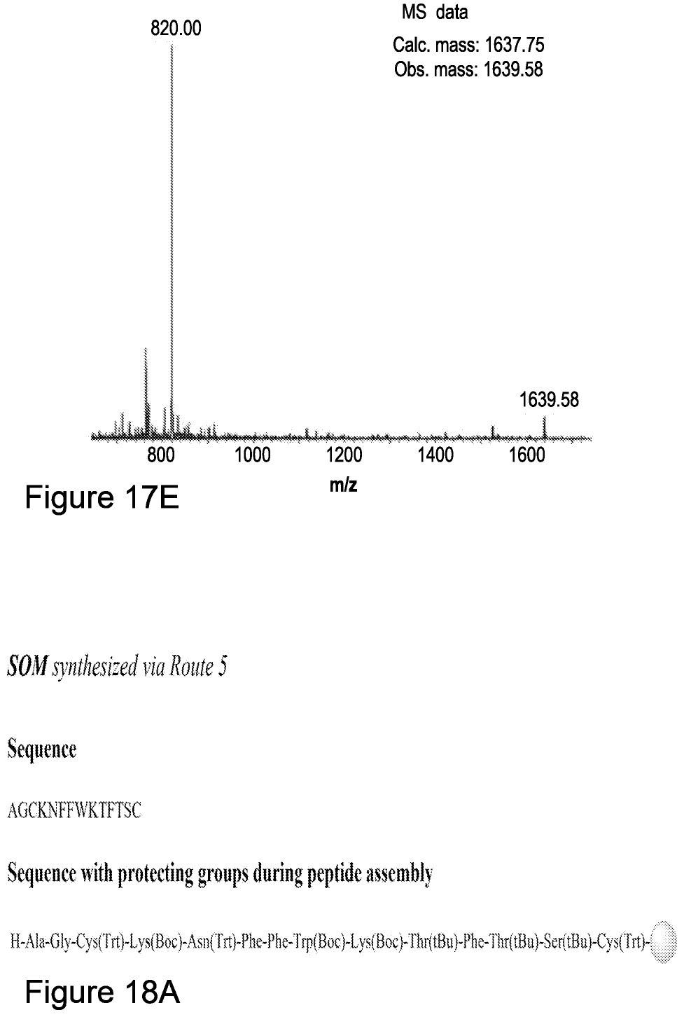

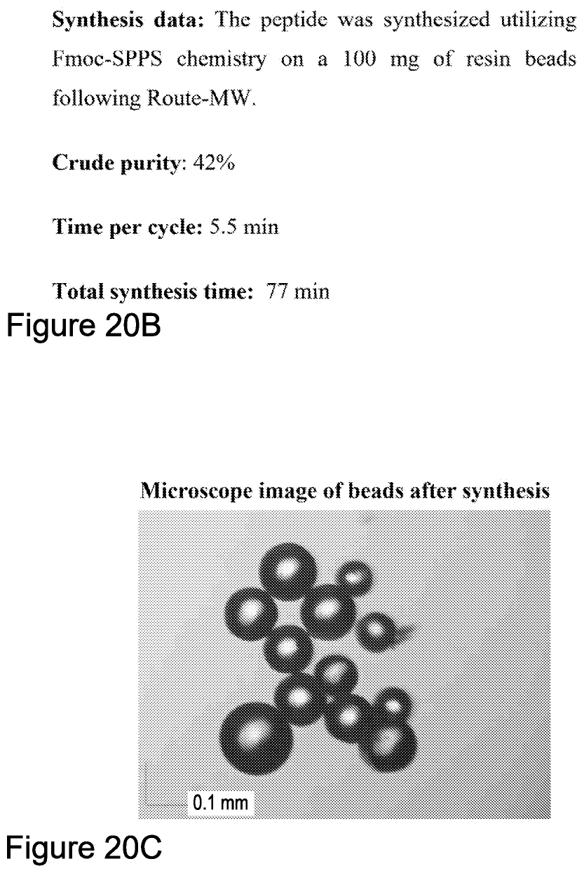

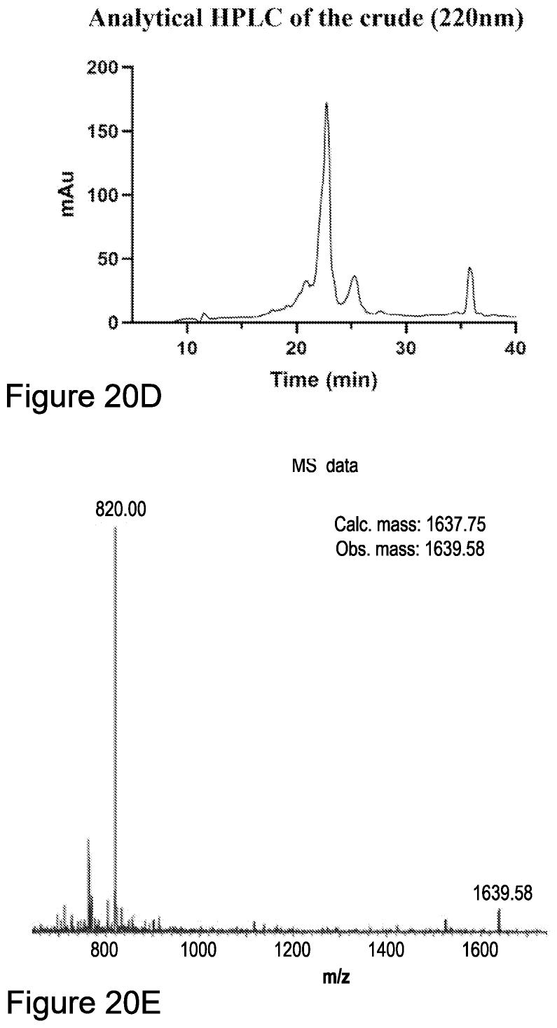

To further push the limits of HTFSPS, the effects of essential parameters were evaluated for the synthesis of a 14-amino acid somatostatin-derived peptide. Somatostatin is an endogenous hormone of the mammalian pituitary gland and is not trivial to synthesize, AGCKNFFWKTFTSC, SEQ ID NO. 5 (Rivier, J.; Kaiser, R.; Galycan, R. Solid-Phase Synthesis of Somatostatin and Glucagon-Selective Analogs in Gram Quantities. Biopolymers 1978, 17, 1927-1938.; Modlin, I. M.; Pavel, M.; Kidd, M.; Gustafsson, B. I. Review Article: Somatostatin Analogues in the Treatment of Gastroenteropancreatic Neuroendocrine (Carcinoid) Tumours. Aliment. Pharmacol. Ther. 2010, 31 (2), 169-188). SOM was synthesized here using automated Mw-SPPS at 90° C. by applying 5 equivalents for couplings periods of at least 2 min (FIGS. 14A-C, Route-Mw; and FIGS. 20A-E). The Mw-SPPS synthesis afforded SOM in a purity of 41% indicating that it is a challenging-to-synthesize peptide even using state-of-the-art methods (FIGS. 14A-C, Route-Mw; and FIGS. 20A-E). Synthesis of SOM (SEQ ID NO. 5) by HTFSPS via Route-3 resulted in a crude purity of 60%, which is significantly higher than by Mw-SPPS (FIGS. 14A-C, Route-3, and FIGS. 16A-E). This proves that HTFSPS surpasses state-of-the-art Mw-SPPS even when lower concentrations of reagents and shorter reaction times are used at almost the same temperature. Since SOM proved efficacy with such a challenging sequence, it was a suitable candidate for evaluating different reaction conditions. Synthesizing SOM at a low temperature via Route-2 did not result in a detectable product (FIGS. 14A-C. Route-2, and FIGS. 15A-E). Given that the only difference between Route-2 and Route-3 is the temperature, it confirms that using elevated temperatures (e.g., 50-90° C. is crucial for accelerating the HTFSPS of hard-to-synthesize peptides like SOM. To examine the effect of the mixing rate, SOM was synthesized via Route-4, which only differs from Route-3 by employing a stirring rate of 100 rpm instead of 1200 rpm (FIGS. 14A-C, Route-4, and FIGS. 17A-E). Synthesis of SOM via Route-4 resulted in a 48% purity, which is significantly lower than the purity gains via Route-3, showing that stirring rate is also an important factor in addition to the temperature (FIGS. 14A-C, Route-4, and FIGS. 17A-E). Interestingly, synthesizing SOM at a low stirring rate via Route-4 was still more beneficial in purity, process time, and reagents quantity, than by Mw-SPPS microwave.

The above results verify, independently, that the effect of both heating and stirring on SOM synthesis outcome is dramatic. It suggests that fast stirring and a high temperature can be used to compensate for low concentration of reagents and/or short the reactions also for peptides that are not easy to synthesize.

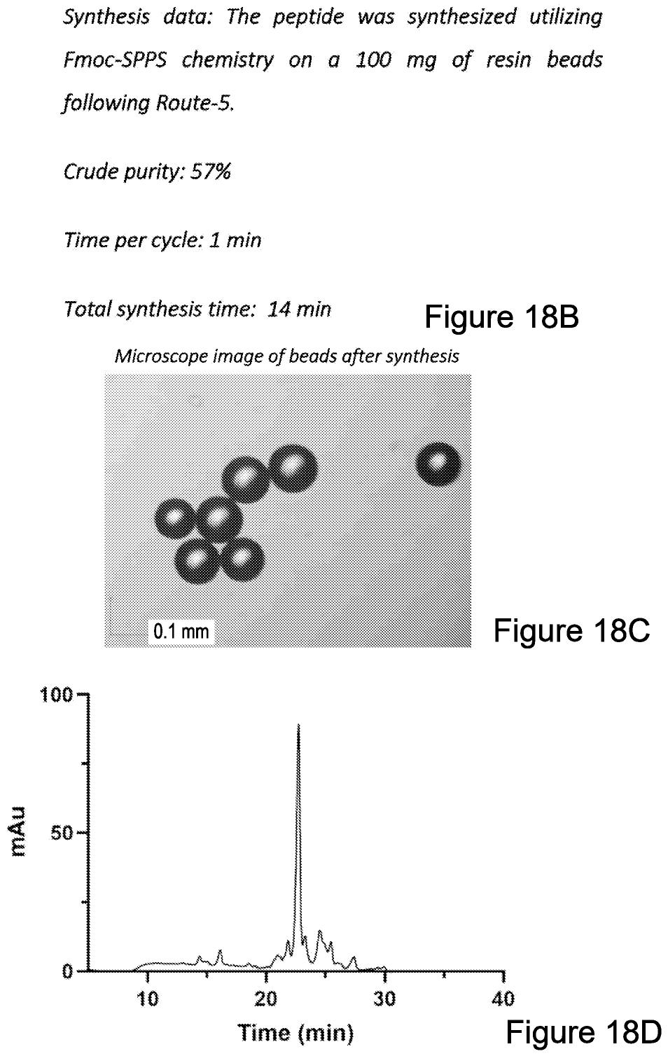

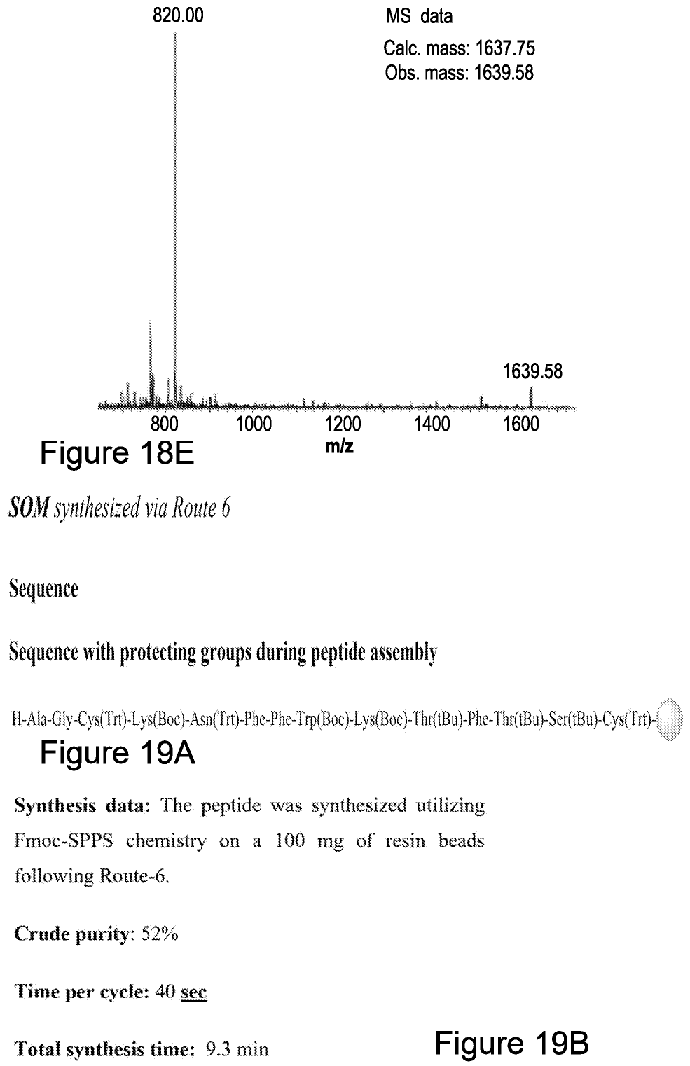

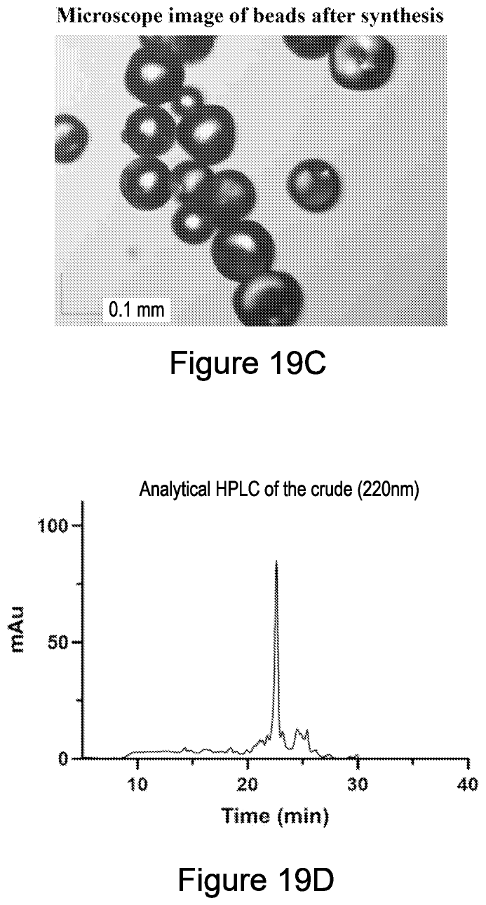

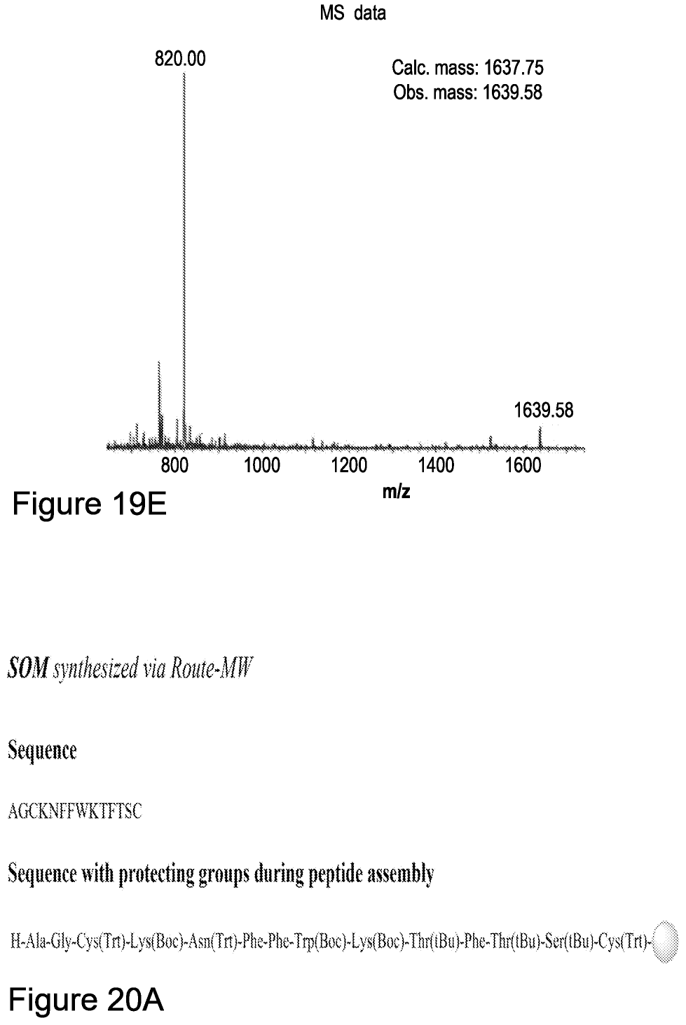

After understanding the influence of fast mixing and a high temperature on HTFSPS efficiency, we wanted to check if even shorter reactions are applicable. SOM (SEQ ID NO. 5) was synthesized via HTFSPS Route-5 (FIGS. 14A-C, Route-5, and FIGS. 18A-E) and Route-6 (FIGS. 14A-C, Route-6, and FIGS. 19A-E), employing short reaction and washings of twenty and ten seconds, respectively. Six equivalents of amino acids and 20% piperidine were used to compensate for the rapid cycles while the stirring rate, solution volumes, and temperature were identical to Route-3. Synthesis of SOM using Route-5 resulted in a crude purity of 57%. which is very close to the one gained using Route-3 (FIGS. 14A-C, Route-5, and FIGS. 18A-E). By performing reaction and washing steps at twenty seconds, the entire SPPS process took ⅓ of the time used for the synthesis via Route-3, whereas the purity was decreased only by 3%. SOM synthesis following Route-6 resulted in a further decrease in purity to 52% (FIGS. 14A-C. Route-6, and FIGS. 19A-E). The synthesis of SOM using Route-6 presents the shortest HTFSPS cycles, as the total time required to introduce each amino acid was 40 seconds, while the payment in purity is reasonable. This proved that a combination of fast stirring, high temperature and slightly increasing the reagent concentrations could compensate for the short reaction time. Comparing SOM synthesis via Route-3 and Route-5 showed that time and concentrations are somehow interchangeable. The reasonable purity obtained in the synthesis of SOM via Route-5 and Route-6 proved that HTFSPS can be applied for fast synthesis of even difficult-to-synthesize peptides. HTFSPS via Route-6 provided accessibility to peptides in record time, offering a reasonably green and cost-efficient strategy.

### Example 6-High-Temperature Fast Stirred Peptide Synthesis (HTFSPS) of a Random Peptide

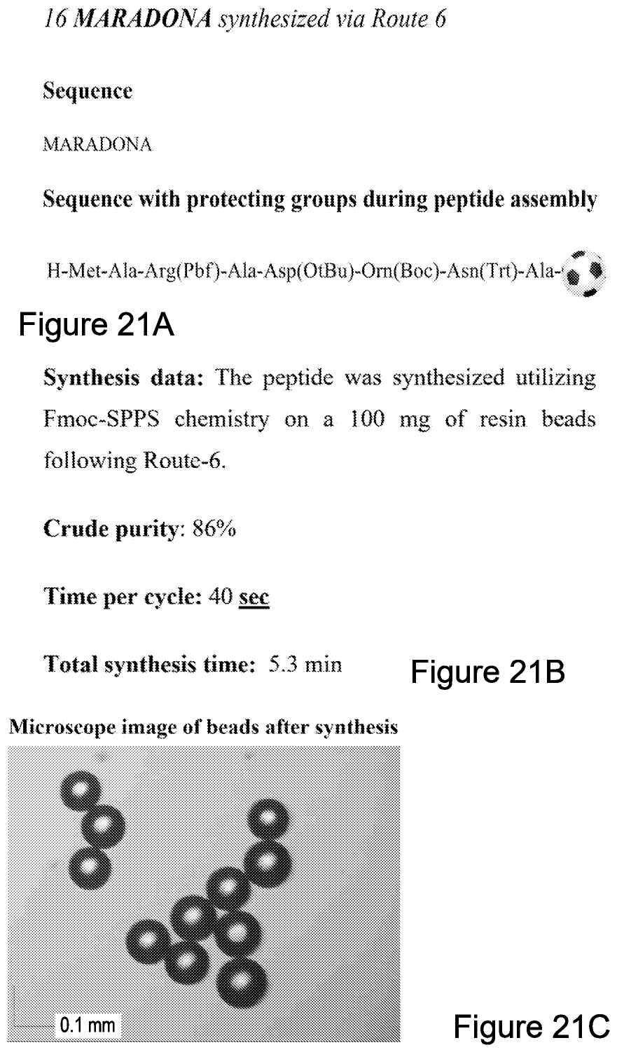

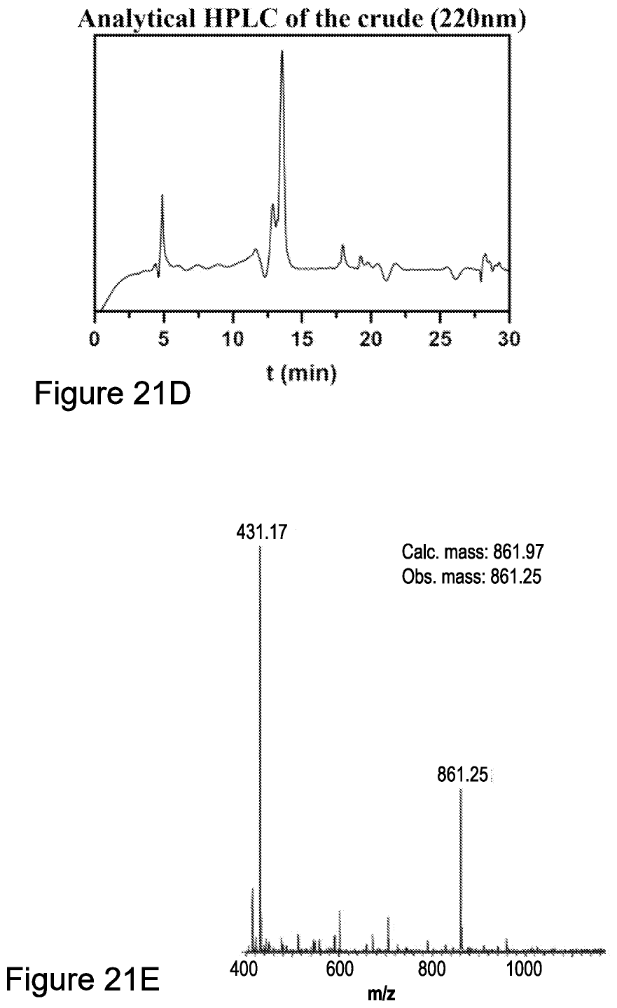

To confirm that short cycles can be used for other peptides, we selected a completely random peptide, MARADONA (SEQ ID NO. 6), and synthesized it via Route-6 in a crude purity of above 80% (FIGS. 21A-E). This model is an important case study since there was no previous knowledge of the complexity or difficulty of the sequence and it is composed of a variety of canonical and non-canonical amino acids. Route-6 protocol was not optimized for MARADONA, yet the octapeptide was obtained in sufficient purity in a process that took less than ten minutes, thus demonstrating the generality of HTFSPS.

The above examples and results indicate that accelerating SPPS can be done by designing a reactor and process that maximizes the contribution of all parameters and not only by employing a high concentration of reagents. HSS-SPPS and HTFSPS are the only methods reported to date which take advantage of fast overhead mixing (over 600 rpm) of both reagents and support for improving peptide synthesis processes. Compared to HSS-SPPS, HTFSPS benefits from the contribution of heating which allows acceleration of the process. High temperature increases diffusion and reaction kinetics, but might also result in side reactions like epimerization and aspartimide formation.

In the examples shown here, these side reactions seem to be subsided in HTFSPS because of the short reactions, the use of low base concentration, and avoiding preactivation at high temperatures (frequently applied in other systems). This suggests that the process can be performed without changing the temperature between steps which is a unique and practical advantage over other setups. Without wishing to be bound by any theory of mechanism of action the ability to decrease reagent excess, shorten reaction time and avoid undesired side reactions benefits from the high efficiency of fast overhead stirring. It is important to note that in each HTFSPS example, the same activator, base, mixing setup were used for all steps of the synthesis. No special additives, solvents, or amino acid protecting groups were used to avoid side products. Unlike fixed-bed setups, beads swelling and size increase during peptide elongation does not pose a limitation in HTFSPS hence enabled the use of a high loading resin. Using high-loading resin allowed maximizing the output from each process, using high reagents concentrations without increasing the molar excess and minimizing the volume of solvents.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-49. (canceled)

50. A method for performing at least one cycle of solid phase synthesis of a peptide, a modified peptide, or a hybrid thereof, the method comprising the steps of:
i) providing a reactor comprising a reaction chamber and a stirring apparatus, wherein the stirring apparatus comprises an impeller having at least two blades rotatable about an axis;
ii) inserting beads of functionalized polymeric resin and at least one solvent into the reactor to provide a reaction mixture, wherein the reaction mixture is in contact with the rotatable blades;
ii) inserting at least one reactant into the reaction chamber; and
iv) spinning the impeller for a period of time, at a rotational rate of at least 600 rounds per minute (rpm), while maintaining a shear rate of at least 3·10³ sec⁻¹, wherein the temperature within the reaction chamber is in the range of 40° C., to 100° C.,
thereby performing at least one cycle of the solid phase synthesis of the peptide, a modified peptide, or a hybrid thereof.

51. The method of claim 50, wherein the process further comprises heating the reaction chamber to allow the temperature range in step (iv).

52. The method of claim 50, wherein step (iv) is performed at a temperature in the range of 50° C., to 90° C.

53. The method of claim 50, wherein the reactor further comprises a heating assembly and an enclosure, which defines an internal cavity and comprises the reaction chamber, wherein the enclosure has an internal face facing the internal cavity and an external face, wherein the at least two blades are disposed within the internal cavity and wherein the heating assembly is in contact with the external enclosure face.

54. The method of claim 53, wherein the stirring apparatus is a mechanical stirrer configured to spin the at least two blades at a rotational rate of 600 to 1400 rpm.

55. The method of claim 53, wherein the heating assembly is surrounding the enclosure, wherein the heating assembly is selected from a circulating fluid bath and a heating jacket.

56. The method of claim 53, wherein the internal cavity is divided by a semipermeable disc to a top internal cavity portion and a bottom internal cavity portion, wherein the semipermeable disc has a plurality of pores therein.

57. The method of claim 56, wherein the semipermeable disc is permeable to fluids and impermeable to the beads of the functionalized polymeric resin.

58. The method of claim 56, wherein the rotatable blades are disposed within the top internal cavity portion.

59. The method of claim 56, wherein the reaction chamber is within the top internal cavity portion, and wherein in step (iv) the shear rate and the temperature range are maintained there within.

60. The method of claim 56, wherein the semipermeable disc is a glass fritted disc.

61. The method of claim 56, wherein the reactor further comprises a conduit extending from a proximal conduit end to a distal conduit end, wherein the proximal conduit end is connected to a portion of the enclosure, which encloses the bottom internal cavity portion, and the distal conduit end is located out of the enclosure.

62. The method of claim 61, wherein the reactor further comprises a valve configured to monitor flow of fluids within the conduit.

63. The method of claim 56, wherein the reactor further comprises a three way bidirectional conduit extending from a proximal conduit end to a first distal conduit end and to a second distal conduit end, wherein the proximal conduit end is connected to a portion of the enclosure, which encloses the bottom internal cavity portion, wherein the first distal conduit end is connected directly or indirectly to an inert gas source, wherein the second distal conduit end is connected directly or indirectly to a vacuum pump, wherein the reactor further comprises a three way valve configured to monitor flow of both liquids and gasses.

64. The method of claim 53, comprising the steps of:
i) providing the reactor;
ii) inserting beads of functionalized polymeric resin and at least one solvent into the enclosure to provide the reaction mixture in contact with the rotatable blades;
iii) inserting at least one reactant into the reaction chamber within the enclosure; and
iv) spinning the impeller for a period of time, at a rotational rate of at least 600 rpm, to maintain the shear rate of at least 3·10³ sec⁻¹ within the reaction chamber, wherein the temperature within the reaction chamber is in the range of 40° C., to 100° C.,
wherein the method further comprises activating the heating assembly to bring the reaction mixture within the enclosure to the temperature range in step (iv).

65. The method of claim 64, comprising the steps of:
i) providing the reactor;
ii) inserting the beads of functionalized polymeric resin into the top internal cavity portion and inserting the at least one solvent into the enclosure, wherein at least a portion of the solvent is in contact with the beads in the top internal cavity portion, to provide the reaction mixture in contact with the rotatable blades;
iii) activating the heating assembly to bring the solvent within the enclosure to a temperature in the range of 40° C., to 100° C.;
iv) inserting at least one reactant into the reaction chamber within the enclosure, wherein the at least one reactant is in contact with the solvent and the beads in the top internal cavity portion, wherein the at least one reactant is in contact with the solvent and the beads in the top internal cavity portion; and
v) inserting inert gas into the enclosure through the conduit;
vi) spinning the impeller for a period of time in the range of 5 seconds to 90 seconds, at a rotational rate of at least 600 rpm, to maintain the shear rate of at least 3·10³ sec⁻¹ within the reaction chamber, wherein the temperature within the reaction chamber is in the range of 4° C., to 100° C.; and
vii) applying vacuum by the vacuum pump to the bottom internal cavity portion, wherein upon the application of vacuum the solvent is substantially evacuated through the conduit, and the beads are maintained in the top internal cavity portion by the semipermeable disc.

66. The method of claim 50, wherein said functionalized beads of polymeric resin comprise coupling capacity of 0.2-1.0 mmol/g or 1.0-3.0 mmol/g.

67. The method of claim 50, for performing a cycle in the solid phase synthesis of a peptide, a modified peptide, or a hybrid thereof, wherein the method comprises the steps of:
(a) providing a reactor comprising a reaction chamber and a stirring apparatus comprising an impeller having at least two blades rotatable about an axis;
(b) inserting beads of functionalized polymeric resin and at least one solvent into the reactor to provide a reaction mixture, wherein the reaction mixture is in contact with the rotatable blades;
(c) inserting at least one protected monomeric organic molecule and at least one coupling agent into the reaction chamber and spinning the impeller, thereby forming a coupling product of the protected monomeric organic molecule and the resin;
(d) washing excess of said protected monomeric organic molecule; and
(e) inserting at least one deprotecting reagent into the reaction chamber and spinning the impeller, thereby removing at least one protecting group from the coupling product, forming a coupling product of the deprotected monomeric organic molecule and the resin, thereby completing a cycle in the solid phase synthesis of a peptide, a modified peptide, or a hybrid thereof,
wherein the spinning of the impeller in at least one of steps (c) and (e) is performed at a temperature in the range of 40° C., to 100° C., for a period of time, at a rotational rate of at least 600 rpm, while maintaining a shear rate of at least 3·10³ sec⁻¹, optionally wherein steps (c) to (e) are repeated a plurality of cycles.

68. The method of claim 67, wherein each one of steps (c) and (e) is performed at a temperature in the range of 40° C., to 100° C.

69. The method of claim 50, wherein the modified peptide or hybrid thereof is a glycopeptide, glycoprotein, or a phosphopeptide.


SEQUENCE LISTING (6 sequences)

SEQ ID NO: 1 - protein, 9 aa, Artificial Sequence
  Synthetic peptide
KLLQDILDA

SEQ ID NO: 2 - protein, 5 aa, Artificial Sequence
  Synthetic peptide
HFGWI

SEQ ID NO: 3 - protein, 9 aa, Homo sapiens
  DISULFID (1)..(6): cyclization
CYFQNCPRG

SEQ ID NO: 4 - protein, 9 aa, Artificial Sequence
  Synthetic peptide
  MISC_FEATURE (1)..(1): D Cystein
  DISULFID (1)..(6): cyclization
  MISC_FEATURE (6)..(6): D Cystein
CYFQNCPRG

SEQ ID NO: 5 - protein, 14 aa, Homo sapiens
  DISULFID (3)..(14): cyclization
AGCKNFFWKTFTSC

SEQ ID NO: 6 - protein, 7 aa, Artificial Sequence
  Synthetic peptide
MARADNA